SEQUENCE LISTING (76 sequences)

SEQ ID NO: 1 - protein, 5 aa, Artificial Sequence
  SYNTHESIZED
INAMN

SEQ ID NO: 2 - protein, 19 aa, Artificial Sequence
  SYNTHESIZED
RIRSKSNNYATYYADSVKD

SEQ ID NO: 3 - protein, 10 aa, Artificial Sequence
  SYNTHESIZED
DITRAGYFDV

SEQ ID NO: 4 - protein, 10 aa, Artificial Sequence
  SYNTHESIZED
SASSSVIYMH

SEQ ID NO: 5 - protein, 7 aa, Artificial Sequence
  SYNTHESIZED
STSNLAS

SEQ ID NO: 6 - protein, 9 aa, Artificial Sequence
  SYNTHESIZED
QQRSSYPLT

SEQ ID NO: 7 - protein, 5 aa, Artificial Sequence
  SYNTHESIZED
TSAMN

SEQ ID NO: 8 - protein, 19 aa, Artificial Sequence
  SYNTHESIZED
RIRSKNYNYATYYADSVKD

SEQ ID NO: 9 - protein, 6 aa, Artificial Sequence
  SYNTHESIZED
PPGFAY

SEQ ID NO: 10 - protein, 11 aa, Artificial Sequence
  SYNTHESIZED
RTSQEISGHLS

SEQ ID NO: 11 - protein, 7 aa, Artificial Sequence
  SYNTHESIZED
AASSLDS

SEQ ID NO: 12 - protein, 9 aa, Artificial Sequence
  SYNTHESIZED
LQYSSYPYT

SEQ ID NO: 13 - protein, 121 aa, Artificial Sequence
  SYNTHESIZED
EVQLVETGGGLVQPKGSLKLSCAVSGITFNINAMNWVRQAPGKGLEWVARIRSKSNNYATYYADSVKDRFTISRDDSQSMLYLQMNNLKTEDTAMYYCVRDITRAGYFDVWGAGTTVTVSS

SEQ ID NO: 14 - protein, 106 aa, Artificial Sequence
  SYNTHESIZED
QIVLTQSPAIMSASPGEKVTITCSASSSVIYMHWFQQKPGTSPKLWIYSTSNLASGVPARFSGSGSGTSYSLTISRMEAEDAATYYCQQRSSYPLTFGAGTKLELK

SEQ ID NO: 15 - protein, 117 aa, Artificial Sequence
  SYNTHESIZED
EVQLVESGGGLVRPKGSLKLSCAASGFTLNTSAMNWVRQVPGKGLEWVGRIRSKNYNYATYYADSVKDRFTIFRDDSQNMLYLQMNNLKTEDTAMYYCVRPPGFAYWGQGTLVTVSA

SEQ ID NO: 16 - protein, 107 aa, Artificial Sequence
  SYNTHESIZED
DIQMTQSPSSLSASLGERVSLTCRTSQEISGHLSWLQQKPDGTIKRLIYAASSLDSGVPKRFSGSRSGSDYSLTISSLESEDFADYYCLQYSSYPYTFGGGTKLEIK

SEQ ID NO: 17 - DNA, 363 bp, Artificial Sequence
  SYNTHESIZED
gaggtgcagcttgttgagactggtggaggattggtgcagcctaaagggtcattgaaactctcatgtgcagtctctggaatcaccttcaatatcaatgccatgaactgggtccgccaggctccaggaaagggtttggaatgggttgctcgcataagaagtaaaagtaataattatgcaacatattatgccgattcagtgaaggacaggttcaccatctccagagatgattcacaaagcatgctctatctgcaaatgaacaacttgaaaactgaggacacagccatgtattactgtgtgagagatataactcgggccgggtacttcgatgtctggggcgcagggaccacggtcaccgtctcctca

SEQ ID NO: 18 - DNA, 318 bp, Artificial Sequence
  SYNTHESIZED
caaattgttctcacccagtctccagcaatcatgtctgcatctccaggggagaaggtcaccataacctgcagtgccagctcaagtgtaatttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttatccgctcacgttcggtgctgggaccaagctggagctgaaa

SEQ ID NO: 19 - DNA, 351 bp, Artificial Sequence
  SYNTHESIZED
gaggtgcagcttgttgagtctggtggaggattggtgcggcctaaagggtcattgaaactctcatgtgcagcctctggattcaccctcaatacctccgccatgaactgggtccgccaggttccaggaaagggtttggaatgggttggtcgcataagaagtaaaaattataattatgcaacatattatgccgattcagtgaaagacaggttcaccattttcagagatgattcacaaaacatgctctatctgcaaatgaacaacttgaaaactgaggacacagccatgtattactgtgtgaggccccccgggtttgcttactggggccaagggactctggtcactgtctctgca

SEQ ID NO: 20 - DNA, 321 bp, Artificial Sequence
  SYNTHESIZED
gacatccagatgacccagtctccatcctccttatctgcctctctgggagaaagagtcagtctcacttgtcggacaagtcaggaaattagtggtcacttaagctggcttcagcagaaaccagatggaactattaaacgcctgatctacgccgcatccagtttagattctggtgtcccaaaaaggttcagtggcagtaggtctgggtcagattattctctcaccatcagcagccttgagtctgaagattttgcagactattactgtctacaatattctagttatccgtacacgttcggaggggggaccaagctggaaataaaa

SEQ ID NO: 21 - protein, 8 aa, Artificial Sequence
  SYNTHESIZED
GGTFSSYA

SEQ ID NO: 22 - protein, 8 aa, Artificial Sequence
  SYNTHESIZED
ISAYNGNT

SEQ ID NO: 23 - protein, 20 aa, Artificial Sequence
  SYNTHESIZED
ARDPPLRYFDWFRGSDWFDP

SEQ ID NO: 24 - protein, 8 aa, Artificial Sequence
  SYNTHESIZED
SGSIASNY

SEQ ID NO: 25 - protein, 3 aa, Artificial Sequence
  SYNTHESIZED
EDN

SEQ ID NO: 26 - protein, 9 aa, Artificial Sequence
  SYNTHESIZED
QSYDSTNGV

SEQ ID NO: 27 - protein, 126 aa, Artificial Sequence
  SYNTHESIZED
QMQLVQSGAEVKKPGSSVKVSCKASGGTFSSYAISWVRQAPGQGLEWMGWISAYNGNTNYAQKLQGRVTMTTDTSTSTAYMELRSLRSDDTAVYYCARDPPLRYFDWFRGSDWFDPWGQGTLVTVS

SEQ ID NO: 28 - protein, 110 aa, Artificial Sequence
  SYNTHESIZED
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSTNGVFGGGTKLTVL

SEQ ID NO: 29 - protein, 8 aa, Artificial Sequence
  SYNTHESIZED
GYTFTDYA

SEQ ID NO: 30 - protein, 8 aa, Artificial Sequence
  SYNTHESIZED
INPGSGNT

SEQ ID NO: 31 - protein, 9 aa, Artificial Sequence
  SYNTHESIZED
ARDRQGPDY

SEQ ID NO: 32 - protein, 6 aa, Artificial Sequence
  SYNTHESIZED
NIGSKN

SEQ ID NO: 33 - protein, 3 aa, Artificial Sequence
  SYNTHESIZED
GDS

SEQ ID NO: 34 - protein, 12 aa, Artificial Sequence
  SYNTHESIZED
QVWDSNSDHPFV

SEQ ID NO: 35 - protein, 115 aa, Artificial Sequence
  SYNTHESIZED
WVQLVESGPEVKKPGASVNVSCKASGYTFTDYAIHWVRQAPGQRPEWMGWINPGSGNTKYSQKFQVRVTITRDTSASTAYMEMTSLRSEDTAIYYCARDRQGPDYWGQGTLVTVS

SEQ ID NO: 36 - protein, 109 aa, Artificial Sequence
  SYNTHESIZED
SYELMQPPSVSMAPGQTARITCGGNNIGSKNVHWYQQKSGQAPVLVVHGDSDRPSGIPERISGRNFGNTATLTINRVEAGDEADYYCQVWDSNSDHPFVFGTGTKVTVL

SEQ ID NO: 37 - protein, 8 aa, Artificial Sequence
  SYNTHESIZED
GGTFSNPV

SEQ ID NO: 38 - protein, 8 aa, Artificial Sequence
  SYNTHESIZED
IITMFGTT

SEQ ID NO: 39 - protein, 9 aa, Artificial Sequence
  SYNTHESIZED
AKDPPMFDY

SEQ ID NO: 40 - protein, 6 aa, Artificial Sequence
  SYNTHESIZED
QSISSY

SEQ ID NO: 41 - protein, 3 aa, Artificial Sequence
  SYNTHESIZED
DAS

SEQ ID NO: 42 - protein, 10 aa, Artificial Sequence
  SYNTHESIZED
QQYDNLLALT

SEQ ID NO: 43 - protein, 115 aa, Artificial Sequence
  SYNTHESIZED
QVQLVQSGAEVKKPGSSVKVSCKSSGGTFSNPVISWVRQAPGQGLEWMGGIITMFGTTYYGNSVRGRFIVSRDNSKNMLFLQMNSLRAEDTAVYYCAKDPPMFDYWGQGTLVTVS

SEQ ID NO: 44 - protein, 108 aa, Artificial Sequence
  SYNTHESIZED
DIQLTQSPSSLSASVGDRVTITCRASQSISSYLNWYQQKPGKAPKLLIYDASNLETGVPSRFSGSGSGTDFTFTISSLQPEDIATYYCQQYDNLLALTFGGGTKVDIK

SEQ ID NO: 45 - protein, 8 aa, Artificial Sequence
  SYNTHESIZED
GGYIDRYF

SEQ ID NO: 46 - protein, 7 aa, Artificial Sequence
  SYNTHESIZED
IYTSGRT

SEQ ID NO: 47 - protein, 9 aa, Artificial Sequence
  SYNTHESIZED
ARDRQGPDY

SEQ ID NO: 48 - protein, 6 aa, Artificial Sequence
  SYNTHESIZED
QGIRND

SEQ ID NO: 49 - protein, 3 aa, Artificial Sequence
  SYNTHESIZED
AAS

SEQ ID NO: 50 - protein, 9 aa, Artificial Sequence
  SYNTHESIZED
LQHNTYPWT

SEQ ID NO: 51 - protein, 114 aa, Artificial Sequence
  SYNTHESIZED
EVQLVETGPGLVKPSQTLSLTCSVSGGYIDRYFIHWVRQAPGQRPEWMGWIYTSGRTKYSQKFQVRVTITRDTSASTAYMEMTSLRSEDTAIYYCARDRQGPDYWGQGTLVTVS

SEQ ID NO: 52 - protein, 107 aa, Artificial Sequence
  SYNTHESIZED
DIVMTQSPSSLSASVGDRVTITCRASQGIRNDLGWYQQKPGKAPKRLIYAASSLQSGVPSRFSGSGSGTEFTLTISSLQPEDFATYYCLQHNTYPWTFGQGTKVEIK

SEQ ID NO: 53 - protein, 21 aa, Artificial Sequence
  SYNTHESIZED
CISKLYGSAGPPPTGEEDTAE

SEQ ID NO: 54 - protein, 20 aa, Artificial Sequence
  SYNTHESIZED
ISKLYGSAGPPPTGEEDTAE

SEQ ID NO: 55 - DNA, 408 bp, Artificial Sequence
  SYNTHESIZED
atgctgttggggctgaagtgggtttgctttgttgttttttatcaaggtgtgcattgtgaggtgcagcttgttgagtctggtggaggattggtgcggcctaaagggtcattgaaactctcatgtgcagcctctggattcaccctcaatacctccgccatgaactgggtccgccaggttccaggaaagggtttggaatgggttggtcgcataagaagtaaaaattataattatgcaacatattatgccgattcagtgaaagacaggttcaccattttcagagatgattcacaaaacatgctctatctgcaaatgaacaacttgaaaactgaggacacagccatgtattactgtgtgaggccccccgggtttgcttactggggccaagggactctggtcactgtctctgca

SEQ ID NO: 56 - DNA, 381 bp, Artificial Sequence
  SYNTHESIZED
atgagggttcctgctcacgtttttggcttcttgttgctctggtttccaggtaccagatgtgacatccagatgacccagtctccatcctccttatctgcctctctgggagaaagagtcagtctcacttgtcggacaagtcaggaaattagtggtcacttaagctggcttcagcagaaaccagatggaactattaaacgcctgatctacgccgcatccagtttagattctggtgtcccaaaaaggttcagtggcagtaggtctgggtcagattattctctcaccatcagcagccttgagtctgaagattttgcagactattactgtctacaatattctagttatccgtacacgttcggaggggggaccaagctggaaataaaa

SEQ ID NO: 57 - protein, 136 aa, Artificial Sequence
  SYNTHESIZED
MLLGLKWVCFVVFYQGVHCEVQLVESGGGLVRPKGSLKLSCAASGFTLNTSAMNWVRQVPGKGLEWVGRIRSKNYNYATYYADSVKDRFTIFRDDSQNMLYLQMNNLKTEDTAMYYCVRPPGFAYWGQGTLVTVSA

SEQ ID NO: 58 - protein, 127 aa, Artificial Sequence
  SYNTHESIZED
MRVPAHVFGFLLLWFPGTRCDIQMTQSPSSLSASLGERVSLTCRTSQEISGHLSWLQQKPDGTIKRLIYAASSLDSGVPKRFSGSRSGSDYSLTISSLESEDFADYYCLQYSSYPYTFGGGTKLEIK

SEQ ID NO: 59 - DNA, 420 bp, Artificial Sequence
  SYNTHESIZED
atgctgttggggctgaagtgggttttctttgttgttttttatcaaggtgtgcattgtgaggtgcagcttgttgagactggtggaggattggtgcagcctaaagggtcattgaaactctcatgtgcagtctctggaatcaccttcaatatcaatgccatgaactgggtccgccaggctccaggaaagggtttggaatgggttgctcgcataagaagtaaaagtaataattatgcaacatattatgccgattcagtgaaggacaggttcaccatctccagagatgattcacaaagcatgctctatctgcaaatgaacaacttgaaaactgaggacacagccatgtattactgtgtgagagatataactcgggccgggtacttcgatgtctggggcgcagggaccacggtcaccgtctcctca

SEQ ID NO: 60 - DNA, 384 bp, Artificial Sequence
  SYNTHESIZED
atgcattttcaagtgcagattttcagcttcctgctaatcagtgcctcagtcataatgtccagaggacaaattgttctcacccagtctccagcaatcatgtctgcatctccaggggagaaggtcaccataacctgcagtgccagctcaagtgtaatttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttatccgctcacgttcggtgctgggaccaagctggagctgaaa

SEQ ID NO: 61 - protein, 140 aa, Artificial Sequence
  SYNTHESIZED
MLLGLKWVFFVVFYQGVHCEVQLVETGGGLVQPKGSLKLSCAVSGITFNINAMNWVRQAPGKGLEWVARIRSKSNNYATYYADSVKDRFTISRDDSQSMLYLQMNNLKTEDTAMYYCVRDITRAGYFDVWGAGTTVTVSS

SEQ ID NO: 62 - protein, 128 aa, Artificial Sequence
  SYNTHESIZED
MHFQVQIFSFLLISASVIMSRGQIVLTQSPAIMSASPGEKVTITCSASSSVIYMHWFQQKPGTSPKLWIYSTSNLASGVPARFSGSGSGTSYSLTISRMEAEDAATYYCQQRSSYPLTFGAGTKLELK

SEQ ID NO: 63 - protein, 12 aa, Artificial Sequence
  SYNTHESIZED
EEKIEWLESHQD

SEQ ID NO: 64 - protein, 12 aa, Artificial Sequence
  SYNTHESIZED
ESHQDADIEDFK

SEQ ID NO: 65 - protein, 12 aa, Artificial Sequence
  SYNTHESIZED
IEDFKAKKKELE

SEQ ID NO: 66 - protein, 12 aa, Artificial Sequence
  SYNTHESIZED
KKELEEIVQPII

SEQ ID NO: 67 - protein, 12 aa, Artificial Sequence
  SYNTHESIZED
VQPIISKLYGSA

SEQ ID NO: 68 - protein, 12 aa, Artificial Sequence
  SYNTHESIZED
LYGSAGPPPTGE

SEQ ID NO: 69 - protein, 12 aa, Artificial Sequence
  SYNTHESIZED
PPTGEEDTAEKD

SEQ ID NO: 70 - protein, 13 aa, Artificial Sequence
  SYNTHESIZED
ISKLYGSAEDTAE

SEQ ID NO: 71 - protein, 10 aa, Artificial Sequence
  SYNTHESIZED
PPTGEEDTAE

SEQ ID NO: 72 - protein, 9 aa, Artificial Sequence
  SYNTHESIZED
  misc_feature (1)..(1): Xaa can be any hydrophobic amino acid or any polar neutral side chain amino acid
  misc_feature (4)..(4): Xaa can be any polar neutral side chain amino acid
XAAXAAAMN

SEQ ID NO: 73 - protein, 23 aa, Artificial Sequence
  SYNTHESIZED
  misc_feature (6)..(6): Xaa can be any polar neutral side chain amino acid
  misc_feature (9)..(9): Xaa can be any hydrophobic amino acid or any polar neutral side chain amino acid
RIRSKXAAXAANYATYYADSNKD

SEQ ID NO: 74 - protein, 30 aa, Artificial Sequence
  SYNTHESIZED
  misc_feature (1)..(1): Xaa can be any naturally occurring amino acid
  misc_feature (4)..(4): Xaa can be any naturally occurring amino acid
  misc_feature (7)..(7): Xaa can be any polar neutral side chain amino acid
  misc_feature (11)..(11): Xaa can be any polar neutral side chain amino acid
  misc_feature (14)..(14): Xaa can be any naturally occurring amino acid
  misc_feature (18)..(18): Xaa can be any hydrophobic or polar neutral side chain amino acid
  misc_feature (21)..(21): Xaa can be any naturally occurring amino acid
  misc_feature (25)..(25): Xaa can be any naturally occurring amino acid
  misc_feature (28)..(28): Xaa can be any naturally occurring amino acid
XAAXAAXAASXAAXAAIXAAXAAHXAAXAA

SEQ ID NO: 75 - protein, 15 aa, Artificial Sequence
  SYNTHESIZED
  misc_feature (1)..(1): Xaa can be any hydrophobic amino acid or any polar neutral side chain amino acid
  misc_feature (4)..(4): Xaa can be any hydrophobic amino acid or any polar neutral side chain amino acid
  misc_feature (8)..(8): Xaa can be any polar neutral side chain amino acid
  misc_feature (12)..(12): Xaa can be any naturally occurring amino acid
XAAXAASXAALXAAS

SEQ ID NO: 76 - protein, 9 aa, Artificial Sequence
  SYNTHESIZED
  misc_feature (1)..(1): Xaa can be any hydrophobic amino acid or any polar neutral side chain amino acid
  misc_feature (3)..(3): Xaa can be any naturally occurring amino acid
  misc_feature (8)..(8): Xaa can be any naturally occurring amino acid
XQXSSYPXT

(19) United States
(12) Patent Application Publication
(10) Pub. No.: US 2022/0259322 A1
(43) Pub. Date: Aug. 18, 2022

(54) ANTI-GRP78 ANTIBODIES AND METHOD OF USE THEREOF

(71) Applicant: Washington University, St. Louis, MO (US)

(72) Inventors: DENNIS HALLAHAN, St. Louis, MO (US); ABHAY KUMAR SINGH, St. Louis, MO (US); VAISHALI KAPOOR, St. Louis, MO (US)

(21) Appl. No.: 17/627,502

(22) PCT Filed: Jul. 16, 2020
(86) PCT No.: PCT/US2020/042374

§ 371 Date: Jan. 14, 2022

Related U.S. Application Data
(60) Provisional application No. 62/874,791, filed on Jul. 16, 2019.

(51) Int. Cl. C07K 16/30; A61N 5/10; A61P 35/00

(52) U.S. Cl. CPC C07K 16/30; A61N 5/10; C07K 2317/622; C07K 2317/56; C07K 2317/34; A61P 35/00

(57) ABSTRACT

The present invention is directed towards isolated antibodies that bind to GRP78. Specifically, compositions comprising anti-GRP78 antigen binding proteins useful in recognition of cancer or tumor cells. Moreover, in some aspects, the anti-GRP78 antigen binding proteins are useful for tumor/cancer-specific delivery of drugs and therapies. In another aspect, the disclosed antigen binding proteins are useful for enhancing radiotherapy in a subject having or suspected of having cancer or a tumor where the antigen binding protein is conjugated to a payload, for example, a therapeutic agent, an imaging agent, or a combination thereof.

### CROSS REFERENCE TO RELATED APPLICATION

This application claims the benefit of U.S. Provisional Application No. 62/874,791, filed Jul. 16, 2019, the disclosure of which is hereby incorporated by reference in its entirety.

### GOVERNMENTAL RIGHTS

This invention was made with government support under CA170169 awarded by the National Institutes of Health. The government has certain rights in the invention.

### FIELD OF TECHNOLOGY

The invention encompasses compositions comprising anti-Grp78 antigen binding proteins useful in recognition of tumor cells and in tumor-specific delivery of drugs and therapies.

### BACKGROUND

In the United States, the probability that an individual, over the course of a lifetime, will develop or die from cancer is 1 in 2 for men and 1 in 3 for women. Therapeutic resistance is a significant barrier in the treatment of cancer and in particular non-small cell lung cancer (NSCLC) and glioblastoma multiforme (GBM). NSCLC ranks among the most frequent cause of cancer related mortality worldwide and remains difficult to cure due to the presentation with advanced stage at the time of diagnosis. In the case of GBM, tumor aggressiveness and recurrence despite multimodal combination therapies result in a median survival of 14 months. Targeted therapy has been a growing topic of investigation to improve therapeutic efficacy for GBM and NSCLC. Antibodies and inhibitors targeting molecules such as VEGFNEGFR-2, EGFR, and RET have been developed to exploit the aberrant protein expression profiles inherent to GBM and NSCLC. However, the marginal improvement in efficacy with these therapeutic approaches highlights the need for discovery of additional molecular targets.

Tumor-specific drug delivery and therapy methods have the potential to reduce or prevent tumor growth in organisms allowing them to lead longer, healthier lives. Many anti-tumor drugs, however, are also toxic to non-tumor cells, resulting in hard to tolerate side-effects. Hence, there is a need in the art for a way to deliver anti-tumor agents specifically to tumor cells to reduce tumor cell growth.

### SUMMARY

One aspect of the present invention encompasses an isolated antibody that binds to 78-kDa glucose-regulated protein (GRP78), where the antibody includes a heavy chain variable domain comprising a CDR1, CDR2, and a CDR3, where the heavy chain variable domain CDR1 comprises SEQ ID NO: 15, the heavy chain variable region domain CDR2 includes SEQ ID NO:5, and the heavy chain variable region domain CDR3 includes SEQ ID NO:11; and a light chain variable domain including a CDR1, CDR2, and CDR3, where the light chain variable domain CDR1 includes SEQ ID NO:25, the light chain variable region domain CDR2 includes SEQ ID NO:19, and the light chain variable region domain CDR3 comprises SEQ ID NO:6. The antibody may recognize an epitope within an amino acid sequence selected from the group consisting of SEQ ID NO:1, SEQ ID NO:2, SEQ ID NO:3, or SEQ ID NO:4. The antibody may be selected from the group consisting of a humanized antibody, a single chain variable fragment (scFv) antibody, an antibody fragment, or a chimeric antibody. The antibody may be conjugated directly or indirectly to a payload selected from the group consisting of a therapeutic agent, an imaging agent, or a combination thereof.

An aspect of the present invention encompasses a method of enhancing radiotherapy in a subject having or suspected of having cancer or a tumor using an antibody that binds to 78-kDa glucose-regulated protein (GRP78), where the antibody is conjugated directly or indirectly to a payload selected from the group consisting of a therapeutic agent, an imaging agent, or a combination thereof, the method involving administering a pharmacologically effective amount of antibody to the subject, such that radiotherapy is enhanced. The method may include administering ionizing radiation to the subject. The method may include imaging the subject. The conjugated therapeutic agent may be an antineoplastic agent.

An aspect of the present invention encompasses a method of imaging cancer or a tumor in a subject in need thereof, using an antibody that binds to 78-kDa glucose-regulated protein (GRP78), where the antibody is conjugated to an imaging agent, the method including administering the conjugated GRP78 to the subject, and imaging cancer in a subject.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts a graph showing the binding of anti-GRP78 mouse monoclonal antibodies to C-terminal GRP78 peptide in an ELISA assay.

FIG. 2 depicts an ELISA assay showing the binding of anti-GRP78 mouse monoclonal antibodies to GRP78 protein.

FIG. 3 shows the cell surface binding of GRP78 antibodies on NSCLC.

FIG. 4 shows the Epitope mapping of GRP78 antibodies (SEQ ID NOs: 63-69). Seven peptides were synthesized from the c-terminus of GRP78. Each peptide was a 12-mer with 5 amino acid overlap. The sequences of the peptides are shown in the figure. Each of the peptide was coated in duplicate on nitrocellulose membrane and incubated with 7A9 antibody. After washing, the blot was developed using chemiluminescence. The arrows indicate positive spot.

FIG. 5A-5B show the epitope mapping of 6F8 antibody. FIG. 5A depicts a graph of the relative response binding. Epitope mapping was performed using Biacore T200. 6F8 was immobilized on the CM5 sensor chip and peptides 86-90 (overlapping peptides in the immunogen region of 6F8) were passed as analyte. Relative binding response (response on blank channel subtracted from the 6F8 immobilized channel) of each peptide to 6F8 was analyzed using the BIAevaluation software. Cut-off for nonbinding peptides is 0 RU. FIG. 5B shows three-dimensional model of GRP78 protein indicating the epitope sequence on the protein in magenta color. The figure was generated using Pymol software (SEQ ID NO: 71).

FIG. 6A-6B shows epitope mapping of 7A9 antibody. FIG. 6A depicts a graph of the relative response binding of 7A9. Epitope mapping was performed using Biacore T200. 7A9 was immobilized on the CM5 sensor chip and peptides 86-90 (overlapping peptides in the immunogen region of 7A9) were passed as analyte. Relative binding response (response on blank channel subtracted from the 7A9 immobilized channel) of each peptide to 7A9 was analyzed using the BIAevaluation software. Cut-off for nonbinding peptides is 0 RU. FIG. 6B shows the three-dimensional model of GRP78 protein indicating the epitope sequence on the protein in red color. The figure was generated using Pymol software (SEQ ID NO: 70).

FIG. 7A-7B show graphs of flowcytometry data for the cell surface binding of GRP78 antibodies. FIG. 7A shows the results of a flow cytometry assay for cell surface binding of 6F8 antibody to A549 cells. Binding conditions: 0.1×10⁶ cells incubated with 3-fold dilutions of antibody started at 2.22 μM. Cells were washed 2× with FACS buffer. Stained with secondary antibody. Washed twice and analyzed in FACS buffer by flow cytometer FACS Canto II (BD). Geometric mean fluorescence intensity was fitted using the “One site-specific binding” in Graphpad Prism software. FIG. 7B shows a graph of the same data as in A fitted using the “Sigmoidal, 4PL, X is log(concentration)” in Graphpad Prism software.

FIG. 8A-8B are scatter plots showing percentage of positive A549 cells. FIG. 8A shows plots 1-7, unstained control and secondary antibody control. FIG. 8B shows plots 8-15.

FIG. 9A-9B are graphs showing the cell surface binding of 6F8 to H460 cells. FIG. 9A shows flow cytometry assay for cell surface binding of 6F8 antibody to H460 cells. Binding conditions: 0.1×106 cells incubated with 3-fold dilutions of antibody started at 0.7 μM. Cells were washed 2× with FACS buffer. Stained with secondary antibody. Washed twice and analyzed in FACS buffer by flow cytometer FACS Canto II (BD). Geometric mean fluorescence intensity was fitted using the One site-specific binding in Graphpad Prism software. FIG. 9B shows a graph of the same data as in A fitted using the “Sigmoidal, 4PL, X is log(concentration)” in Graphpad Prism software.

FIG. 10 shows scatter plots of the percentage of positive H460 cells stained with 3-fold serial dilutions (Plots 1-6) of 6F8 antibody. The scatter plots are colored by fluorescence intensity of fluorochrome used for detection.

FIG. 11A-11B show flow cytometry for cell surface binding of 7A9 antibody to H460 cells. FIG. 11A is the flow cytometry assay for cell surface binding of 7A9 antibody to H460 cells. Binding conditions: 0.1×10⁶ cells incubated with 3-fold dilutions of antibody started at 1.0 μM. Cells were washed 2× with FACS buffer. Stained with secondary antibody. Washed twice and analyzed in FACS buffer by flow cytometer FACS Canto II (BD). Geometric mean fluorescence intensity was fitted using the One site-specific binding in Graphpad Prism software. FIG. 11B shows a graph of the same data as in A fitted using the “Sigmoidal, 4PL, X is log(concentration)” in Graphpad Prism software.

FIG. 12 shows scatter plots of the percentage of positive H460 cells stained with 3-fold serial dilutions (Plots 1-7) of 7A9 antibody. The scatter plots are colored by fluorescence intensity of fluorochrome used for detection.

FIG. 13 shows whole body NIR imaging with GRP78 monoclonal antibodies (un-irradiated tumors) A549 tumors were injected in hind limbs of nude mice. 6F8 and 7A9 antibodies were labeled with the IR dye 800 (Licor). 40 ug of each antibody was injected in the tail vein and imaged every day using the Pearl imager.

FIG. 14A-14C show the biodistribution of the GRP78 monoclonal antibodies by NIR imaging (un-irradiated tumors). FIG. 14A shows the images of the harvested A549 tumors. FIG. 14B shows a bar graph of the signal intensity per gram of the harvested tumors. FIG. 14C shows the signal intensity of all organs per gm of their respective weights.

FIG. 15 shows the internalization of the GRP78 monoclonal antibodies in A549 cells. 6F8 and 7A9 were labeled with pHRodo red pH-sensitive dye that fluoresces red in acidic cellular compartments.

FIG. 16 shows a dot blot showing GRP78 scFv-Fc binding to GRP78 full length protein. For the dot blot, recombinant GRP78 full length protein was spotted onto nitrocellulose membrane in duplicate. After blocking, the blot was incubated with anti GRP78 scFv Fcs 1171 and 1183. The scFv-Fcs were detected for binding to the recombinant GRP78 protein using anti human Fc HRP conjugated antibody.

FIG. 17 shows biacore analysis for binding affinity of anti-GRP78 scFV 1171-Fc to recombinant full length GRP78 protein. The highest concentration of scFv1171-Fc was 500 nM followed by two-fold serial dilutions. The on-, off-rates and dissociation constant are shown in the table.

FIG. 18 shows biacore analysis for binding affinity of anti-GRP78 scFV 1171-Fc to recombinant full length GRP78 protein. The highest concentration of scFv1183-Fc was 500 nM followed by two-fold serial dilutions. The on-, off-rates and dissociation constant are shown in the table.

FIG. 19 shows flow cytometry for cell surface binding of scFv-Fc1171. A549 cells were either sham or irradiated with 3 doses of 3Gy. Cells were harvested and incubated with indicated concentrations of the scFV-Fc1171 antibodies. Representative overlay histograms (blue: secondary antibody control; red: scFv-Fc1171) are shown.

FIG. 20 shows a flow cytometry assay for cell surface binding of scFv-Fc1171 antibody to irradiated A549 cells. Binding conditions: 0.1×106 cells incubated with 4-fold dilutions of antibody started at 100 nM. Cells were washed 2× with FACS buffer. Stained with secondary antibody. Washed twice and analyzed in FACS buffer by flow cytometer FACS Canto II (BD). Geometric mean fluorescence intensity was fitted using the One site-specific binding in GraphpadPrism software. Graph showing the percentage of positive cells fitted using the “Sigmoidal, 4PL, X is log(concentration)” in GraphpadPrism software.

FIG. 21 shows flow cytometry for cell surface binding of Fc negative control. A549 cells were either sham or irradiated with 3 doses of 3Gy. Cells were harvested and incubated with indicated concentrations of the Fc negative control. Representative overlay histograms (blue: secondary antibody control; red: Fc negative control) are shown.

FIG. 22 shows endocytosis of scFv-Fc 1171 in A549 cells. A549 cells were either sham or irradiated with 3 doses of 3Gy. scFv-Fc 1171 was labeled with pHRodo red pH-sensitive dye that fluoresces red in acidic cellular compartments. White arrows indicate the internalized antibody.

FIG. 23 shows dose response of GRP78 scFv-Fc 1171 and Fc using colony formation assay. Cells were seeded and irradiated with 2Gy the following day. scFv-Fc 1171 or the Fc control was added at 2 different concentrations. Another dose of 2Gy was given the next day. Colonies were counted and surviving fraction plotted. Two-way ANOVA was used for statistical analysis. *p<0.05, **p<0.01.

FIG. 24 shows dose response of GRP78 scFv-Fc 1171 and Fc using colony formation assay on H460 cells. Cells were seeded and irradiated with 2Gy the following day. scFv-Fc 1171 or the Fc control was added at 2 different concentrations. Another dose of 2Gy was given the next day. Colonies were counted and surviving fraction plotted. A decreasing trend in surviving fraction is observed with scFvFc1171 in combination with radiation.

FIG. 25 shows the nucleotide and amino acid sequence for the 6F8 antibody. (SEQ ID NOs: 55-58).

FIG. 26 shows the nucleotide and amino acid sequence for the 7A9 antibody. (SEQ ID NOs: 59-62).

FIG. 27 shows the amino acid sequence for the SCFV GRP78-1183 antibody. (SEQ ID NOs: 27-28).

FIG. 28 shows the amino acid sequence for the SCFV GRP78-1164 antibody. (SEQ ID NOs: 35-36).

FIG. 29 shows the amino acid sequence for the SCFV GRP78-1171 antibody. (SEQ ID NOs: 43-44).

FIG. 30 shows the amino acid sequence for the SCFV GRP78-1256 antibody. (SEQ ID NOs: 51-52).

### DETAILED DESCRIPTION

The present disclosure encompasses antigen binding proteins that recognize tumor cells. The disclosure also provides methods of use of the antigen binding proteins disclosed herein. The antigen binding proteins may be used to provide tumor-specific delivery, for instance, of drugs or therapeutic agents, as well as enhancing the efficacy of radiotherapy. In one aspect, the disclosure provides antigen binding proteins which are useful for imaging cancer in a subject. In another aspect, the disclosure provides a method of enhancing radiotherapy in a subject using an antigen binding proteins of the disclosure. Advantageously, these antigen binding proteins specifically bind tumor cells and not normal cells. Additionally, the disclosure provides antigens for the purpose of preparing antigen binding proteins as taught herein.

In an exemplary embodiment, antigen binding proteins of the disclosure specifically bind to epitopes exposed on irradiated cancer or tumor related cells. For instance, antibodies of the disclosure may bind to extracellular, transmembrane or intracellular epitopes on irradiated cancer or tumor related cells. In particular, the present disclosure provides for antigen binding proteins that bind to 78-kDa glucose-regulated protein (GRP78). GRP78 is a multifunctional protein folding chaperone and co-receptor that is highly expressed on the surface of GBM and NSCLC, holds significant promise as a cancer-specific target. As shown herein, non-small cell lung cancer (NSCLC) and glioblastoma multiforme (GBM) cancer cell lines were treated with anti-GRP78 antibodies and evaluated for proliferation, colony formation, cell death, and PI3K/Akt/mTOR signaling. GBM and NSCLC cells treated with anti-GRP78 antibodies showed attenuated cell proliferation, colony formation, and enhanced apoptosis. GBM and NSCLC cells treated with anti-GRP78 antibodies also showed global suppression of PI3K/Akt/mTOR signaling. The efficacy of anti-GRP78 antibodies on tumor growth in combination with ionizing radiation (XRT) was determined in vivo in mouse xenograft models. Combining antibody with XRT resulted in significant tumor growth delay in both NSCLC and GBM heterotopic tumor models. Antibodies targeting GRP78 exhibited antitumor activity and enhanced the efficacy of radiation in NSCLC and GBM both in vitro and in vivo. These data suggest anti-GRP78 antigen binding proteins are effective as a cancer therapy alone or in combination with XRT. The antigen binding proteins and method of using the same are described in further detail below.

### I. Anti-GRP78 Antigen Binding Proteins

In an aspect, GRP78 antigen binding proteins as described herein include those antigen binding proteins which specifically binds GRP78 and have been isolated, characterized, purified, are functional and have been recovered (obtained) for use in a functional therapeutic composition which is administered to a living subject having or suspected of having cancer or a tumor. In an aspect, GRP78 antigen binding proteins as described herein include those antigen binding proteins which specifically binds GRP78 and have been isolated, characterized, purified, are functional and have been recovered (obtained) for use in a functional imaging composition which is administered to a living subject having or suspected of having cancer or a tumor. In another aspect, antigen binding proteins useful herein include those antigen binding proteins which have been isolated, characterized, purified, are functional and have been recovered (obtained) for use in an assay to detect GRP78 in a biological sample obtained from a living subject and detect the development of a cancer or tumor in the subject. In another aspect, antigen binding proteins useful herein include those antigen binding proteins which have been isolated, characterized, purified, are functional and have been recovered (obtained) for use and are listed in Table B, as well as variants thereof (e.g. humanized forms, chimeric forms, and immunological fragments).

The 78-kDa glucose-regulated protein GRP78 (Uniprot ID P11021), also known as BiP and HSP5a, is a multifunctional protein. At the transcription level, GRP78 is encoded by the gene Hsp5a. GRP78 participates in a well-known role in the unfolded protein response (UPR) which is activated after endoplasmic reticulum (ER) stress in the cells. However, GRP78 participates in other activities which depend on its position within the cell. GRP78 is located mainly in the ER, but it has also been observed in the cytoplasm, the mitochondria, the nucleus, the plasma membrane, and secreted, although it is dedicated mostly to engage endogenous cytoprotective processes. Hence, GRP78 may control either UPR and macroautophagy or may activated phosphatidylinositol 3-kinase (PI3K)/AKT pro-survival pathways. GRP78 influences how tumor cells survive, proliferate, and develop chemoresistance.

The phrase “specifically binds” herein means antigen binding proteins bind to GRP78 with an affinity constant or affinity of interaction (KD) of less than 300 nM, less than 250 nM, less than 200 nM, less than 150 nM, less than 100 nM, less than 75 nM, less than 50 nM, less than 25 nM, less than 20 nM, less than 15 nM, less than 10 nM, less than 5 nM, or less than 1 nM.

The term “antigen binding protein” refers to any form of antibody or fragment thereof that exhibits the desired biological activity. Thus, it is used in the broadest sense and specifically covers monoclonal antibodies (including full length monoclonal antibodies), polyclonal antibodies, multispecific antibodies (e.g. bispecific antibodies), and antibody fragments so long as they exhibit the desired biological activity, for example, specifically binding GRP78.

The term “antibody” includes the term “monoclonal antibody”. The term “monoclonal antibody” refers to an antibody that is derived from a single copy or clone, including e.g., any eukaryotic, prokaryotic, or phage clone. Monoclonal antibodies are obtained from a population of substantially homogeneous antibodies, i.e., the individual antibodies comprising the population are identical except for possible naturally occurring mutations or post-translational modification that may be present in minor amounts. Monoclonal antibodies are highly specific, being directed against a single antigenic epitope. “Monoclonal antibody” is not limited to antibodies produced through hybridoma technology. Monoclonal antibodies can be produced using e.g., hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

The term “fragment thereof” encompasses a fragment or a derivative of an antibody that still substantially retain its biological activity. Therefore, the term “antibody fragment” or “fragment thereof” refers to a portion of a full length antibody, generally the antigen binding or variable region thereof. Examples of an immunologically effective fragment thereof include Fab, Fab′, F(ab′)2 and Fv fragments, diabodies, linear antibodies, single-chain molecules, and multispecific antibodies formed from antibody fragments. In some embodiments, the antibody fragments as disclosed herein include, in non-limiting examples, fusions to a Fc domain, a cytokine, a toxin, or an enzyme. In some contexts herein, fragments will be mentioned specifically for emphasis; nevertheless, it will be understood that regardless of whether fragments are specified, the term “antibody” includes such fragments.

Also included within the definition “antibody” for example are single chain forms, generally designated Fv, regions, of antibodies with this specificity. These scFvs are comprised of the heavy and light chain variable regions connected by a linker. In most instances, but not all, the linker may be a peptide. A linker peptide is preferably from about 10 to 25 amino acids in length. Preferably, a linker peptide is rich in glycine, as well as serine or threonine. scFvs can be used to facilitate phage display or can be used for flow cytometry, immunohistochemistry, or as targeting domains. Methods of making and using scFvs are known in the art. In a preferred embodiment, the scFvs of the present disclosure are conjugated to a human constant domain. In some embodiments, the heavy constant domain is derived from an IgG domain, such as IgG1, IgG2, IgG3, or IgG4. In other embodiments, the heavy chain constant domain may be derived from IgA, IgM, or IgE.

The term “antibody” also includes bispecific monoclonal antibodies (i.e. a protein that comprises fragments of two different monoclonal antibodies and consequently binds two different antigens). A specific example of a bispecific monoclonal antibody may be a Bi-specific T-cell Engager (BiTE) which is a fusion protein consisting of two single-chain variable fragments (scFvs) of different antibodies. In certain embodiments, BiTEs from a link between T cells and cancer cells. Accordingly, one scFv is a specific for GRP78 and one scFv binds a T cell. Additionally, an antibody of the disclosure may be a chimeric antigen receptor (CAR), also referred to as an artificial T cell receptor, a chimeric T cell receptor, or a chimeric immunoreceptor. CARs are engineered receptors, which graft an arbitrary specificity onto an immune effector cell. In some embodiments, the CARs as disclosed herein are expressed in immune effector cells. The term “immune effector cell,” as used herein, are cells that are actively involved in the destruction of tumor or cancer cells, e.g., possess anti-tumor/anti-cancer activity. These cells may include, but are not limited to, macrophage cells, lymphocyte cells, natural killer (NK) cells, cytotoxic T cells, and memory T cells. The term “chimeric antigen receptor (CAR)-bearing immune effector cells are immune effector cells that express a chimeric antigen receptor. These cells may include, but are not limited to, CAR-macrophage, CAR-T cells or CAR-bearing iNKT cells (iNKT-CAR). Typically, these CARs are used to graft the specificity of a anti-GRP78 antigen binding protein to an immune effector cell.

Further still, an antibody of the disclosure may be a Dual-affinity Re-targeting Antibody (DART). The DART format is based on the diabody format that separates cognate variable domains of heavy and light chains of the 2 antigen binding specificities on 2 separate polypeptide chains. Whereas the 2 polypeptide chains associate noncovalently in the diabody format, the DART format provides additional stabilization through a C-terminal disulfide bridge. DARTs can be produced in high quantity and quality and reveal exceptional stability in both formulation buffer and human serum. Additionally, included within the definition “antibody” are single-domain antibodies, generally designated sdAb, which is an antibody fragment consisting of a single monomeric variable antibody domain. A sdAb antibody may be derived from camelids (VHH fragments) or cartilaginous fishes (VNAR fragments). As long as the protein retains the ability specifically to bind its intended target, it is included within the term “antibody.”

Preferably, but not necessarily, the antibodies useful in the discovery are produced recombinantly, as manipulation of the typically murine or other non-human antibodies with the appropriate specificity is required in order to convert them to humanized form. Antibodies may or may not be glycosylated, though glycosylated antibodies are preferred. Antibodies are properly cross-linked via disulfide bonds, as is known. An antibody of the disclosure can be modified to optimize or minimize effector function. Further, an antibody of the disclosure can be modified to extend half-life. Still further, an antibody of the disclosure can be modified to improve binding affinity. Methods of modifying an antibody to improve the aforementioned characteristics are known in the art.

The basic antibody structural unit of an antibody useful herein comprises a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acid sequences primarily responsible for antigen recognition. The carboxy-terminal portion of each chain defines a constant region primarily responsible for effector function.

Light chains are classified as gamma, mu, alpha, and lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acid sequences, with the heavy chain also including a “D” region of about 10 more amino acid sequences.

The variable regions of each light/heavy chain pair form the antibody binding site. Thus, an intact antibody has two binding sites, although recombinant versions can be of higher valency. The chains exhibit the same general structure of relatively conserved framework regions (FR) joined by three hypervariable regions, also called complementarity determining regions (hereinafter referred to as “CDRs”). The CDRs from the two chains are aligned by the framework regions, enabling binding to a specific epitope. From N-terminal to C-terminal, both light and heavy chains comprise the domains FR1, CDR1, FR2, CDR2, FR3, CDR3 and FR4 respectively. The assignment of amino acid sequences to each domain is in accordance with known conventions (See, Kabat “Sequences of Proteins of Immunological Interest” National Institutes of Health, Bethesda, Md., 1987 and 1991; Chothia, et al, J. Mol. Bio. (1987) 196:901-917; Chothia, et al., Nature (1989) 342:878-883). For example, Kabat, Chothia, combinations thereof, or other known methods of determining CDRs may be used.

In an aspect, antibodies of the invention are generated with appropriate specificity by standard techniques of immunization of mammals, forming hybridomas from the antibody-producing cells of said mammals or otherwise immortalizing them, and culturing the hybridomas or immortalized cells to assess them for the appropriate specificity. In the present case, such antibodies may be generated by immunizing a human, rabbit, rat or mouse, for example, with a peptide representing an epitope encompassing a region of the GRP78 protein coding sequences or an appropriate subregion thereof. Materials for recombinant manipulation may be obtained by retrieving the nucleotide sequences encoding the desired antibody from the hybridoma or other cell that produces it. These nucleotide sequences may then be manipulated and isolated, characterized, purified and recovered to provide them in humanized form, if desired.

As used herein “humanized antibody” includes an anti-GRP78 antibody that is composed partially or fully of amino acid sequences derived from a human antibody germline by altering the sequence of an antibody having non-human complementarity determining regions (“CDR”). The simplest such alteration may consist simply of substituting the constant region of a human antibody for the murine constant region, thus resulting in a human/murine chimera which may have sufficiently low immunogenicity to be acceptable for pharmaceutical use. Preferably, however, the variable region of the antibody and even the CDR is also humanized by techniques that are by now well known in the art. The framework regions of the variable regions are substituted by the corresponding human framework regions leaving the non-human CDR substantially intact, or even replacing the CDR with sequences derived from a human genome. CDRs may also be randomly mutated such that binding activity and affinity for GRP78 is maintained or enhanced in the context of fully human germline framework regions or framework regions that are substantially human. Substantially human frameworks have at least 90%, 95%, or 99% sequence identity with a known human framework sequence. Fully useful human antibodies are produced in genetically modified mice whose immune systems have been altered to correspond to human immune systems. As mentioned above, it is sufficient for use in the methods of this discovery, to employ an immunologically specific fragment of the antibody, including fragments representing single chain forms.

Further, as used herein the term “humanized antibody” refers to an anti-GRP78 antibody comprising a human framework, at least one CDR from a nonhuman antibody, and in which any constant region present is substantially identical to a human immunoglobulin constant region, i.e., at least about 85-90%, preferably at least 95% identical. Hence, all parts of a humanized antibody, except possibly the CDRs, are substantially identical to corresponding pairs of one or more native human immunoglobulin sequences.

If desired, the design of humanized immunoglobulins may be carried out as follows. When an amino acid sequence falls under the following category, the framework amino acid sequence of a human immunoglobulin to be used (acceptor immunoglobulin) is replaced by a framework amino acid sequence from a CDR-providing nonhuman immunoglobulin (donor immunoglobulin): (a) the amino acid sequence in the human framework region of the acceptor immunoglobulin is unusual for human immunoglobulin at that position, whereas the corresponding amino acid sequence in the donor immunoglobulin is typical for human immunoglobulin at that position; (b) the position of the amino acid sequence is immediately adjacent to one of the CDRs; or (c) any side chain atom of a framework amino acid sequence is within about 5-6 angstroms (center-to-center) of any atom of a CDR amino acid sequence in a three dimensional immunoglobulin model (Queen, et al., op. cit., and Co, ct al, Proc. Natl. Acad. Sci. USA (1991) 88:2869). When each of the amino acid sequences in the human framework region of the acceptor immunoglobulin and a corresponding amino acid sequence in the donor immunoglobulin is unusual for human immunoglobulin at that position, such an amino acid sequence is replaced by an amino acid sequence typical for human immunoglobulin at that position.

The antibodies of the present disclosure may also be conjugated to a payload, such as a therapeutic agent, a detectable label, and/or a delivery device (including, but not limited to, a liposome or a nanoparticle) containing the drug or detectable label. Methods of conjugating an antibody to a therapeutic agent, a detectable label, a liposome, a nanoparticle or other delivery device are known in the art. Generally speaking, the conjugation should not interfere with the antibody recognizing its target, and should not interfere with the active site of the target. In some instances, an antibody may be generated with a cleavable linkage between the antibody and the payload. Such a linker may allow release of the payload at a specific cellular location. Suitable linkers include, but are not limited to, amino acid chains and alkyl chains functionalized with reactive groups for conjugating to both the antibody of the disclosure and the detectable label and/or therapeutic agent.

Anti-GRP78 antigen binding proteins useful herein also include all antigen binding proteins that specifically bind GRP78 in a biological sample. In an exemplary embodiment, an antigen binding protein useful herein include all antigen binding proteins that specifically bind GRP78 present in a biological sample.

In some embodiments, an antigen binding protein of the present invention is conjugated to a therapeutic agent. The therapeutic agent preferably reduces or interferes with cancer or tumor growth or otherwise reduces the effect of the cancer or tumor within the body or organism. A therapeutic agent that reduces the symptoms produced by the cancer or tumor or reduces cancer or tumor growth is suitable for the present disclosure.

Additionally, a therapeutic agent that reduces the symptoms associated with cancer or tumor cell growth will work for purposes of the present disclosure. Non-limiting examples of therapeutic agents may include CAR-bearing immune effector cells, drugs, therapeutic compounds, genetic materials, metals (such as radioactive isotopes), proteins, peptides, carbohydrates, lipids, steroids, nucleic acid based materials, or derivatives, analogues, or combinations thereof in their native form or derivatized with hydrophobic or charged moieties to enhance incorporation or adsorption into a cell. Such therapeutic agents may be water soluble or may be hydrophobic. Non-limiting examples of therapeutic agents may include immune-related agents, thyroid agents, respiratory products, antineoplastic agents, anti-helmintics, anti-malarials, mitotic inhibitors, hormones, anti-protozoans, anti-tuberculars, cardiovascular products, blood products, biological response modifiers, anti-fungal agents, vitamins, peptides, anti-allergic agents, anti-coagulation agents, circulatory drugs, metabolic potentiators, anti-virals, anti-anginals, antibiotics, anti-inflammatories, anti-rheumatics, narcotics, cardiac glycosides, neuromuscular blockers, sedatives, local anesthetics, general anesthetics, or radioactive atoms or ions. Non-limiting examples of therapeutic agents are included in Table A below. An isolated antigen binding peptide of the present disclosure may be conjugated to one, two, three, four, or five therapeutic agents. Methods of conjugating an antibody to a therapeutic agent are known in the art. Generally speaking, the conjugation should not interfere with the antibody recognizing its target, and should not interfere with the active site of the target. In some instances, a scFv may be generated with a cleavable linkage between the scFv and therapeutic agent. Such a linker may allow release of the therapeutic agent at a specific cellular location.

TABLE A
Non-limiting Examples of Therapeutic Agents

| Exemplary Therapeutic | |
|---|---|
| Agent | Non-limiting examples |
| Immune-related agents | immune serums, antitoxins, antivenoms bacterial |
| | vaccines, viral vaccines, rabies prophylaxis products |
| thyroid agents | iodine products and anti-thyroid agents |
| respiratory products | xanthine derivatives theophylline and aminophylline |
| antineoplastic agents | platinum compounds (e.g., spiroplatin, cisplatin, and |
| | carboplatin), methotrexate, fluorouracil, adriamycin, |
| | mitomycin, ansamitocin, bleomycin, cytosine |
| | arabinoside, arabinosyl adenine, mercaptopolylysine, |
| | vincristine, busulfan, chlorambucil, melphalan (e.g., |
| | PAM, L-PAM or phenylalanine mustard), |
| | mercaptopurine, mitotane, monomethyl auristatin E |
| | (MMAE), drug maytansinoids (e.g. DM1), |
| | procarbazine hydrochloride dactinomycin |
| | (actinomycin D), daunorubicin hydrochloride, |
| | doxorubicin hydrochloride, paclitaxel and other |
| | taxenes, rapamycin, manumycin A, TNP-470, |
| | plicamycin (mithramycin), aminoglutethimide, |
| | estramustine phosphate sodium, flutamide, |
| | leuprolide acetate, megestrol acetate, tamoxifen |
| | citrate, testolactone, trilostane, amsacrine (m- |
| | AMSA), asparaginase (L-asparaginase) Erwina |
| | asparaginase, interferon α-2a, interferon α-2b, |
| | teniposide (VM-26), vinblastine sulfate (VLB), |
| | vincristine sulfate, bleomycin sulfate, hydroxyurea, |
| | procarbazine, and dacarbazine |
| anti-helmintics | pyrantel pamoate, piperazine, tetrachloroethylene, |
| | thiabendazole, niclosamide |
| antimalarials | Chloroquine, amodiaquine, antifolate drugs, |
| | proguanil (chloroguanide), mefloquine, quinine, |
| | halofantrine, artemesinin and derivaties, primaquine, |
| | doxycycline, tetracycline, and clindamycin |
| mitotic inhibitors | etoposide, colchicine, and the vinca alkaloids |
| hormones | androgens, progestins, estrogens and antiestrogens, |
| | growth hormone, melanocyte stimulating hormone, |
| | estradiol, beclomethasone dipropionate, |
| | betamethasone, betamethasone acetate and |
| | betamethasone sodium phosphate, vetamethasone |
| | disodium phosphate, vetamethasone sodium |
| | phosphate, cortisone acetate, dexamethasone, |
| | dexamethasone acetate, dexamethasone sodium |
| | phosphate, flunisolide, hydrocortisone, |
| | hydrocortisone acetate, hydrocortisone cypionate, |
| | hydrocortisone sodium phosphate, hydrocortisone |
| | sodium succinate, methylprednisolone, |
| | methylprednisolone acetate, methylprednisolone |
| | sodium succinate, paramethasone acetate, |
| | prednisolone, prednisolone acetate, prednisolone |
| | sodium phosphate, prednisolone tebutate, |
| | prednisone, triamcinolone, triamcinolone acetonide, |
| | triamcinolone diacetate, triamcinolone hexacetonide, |
| | fludrocortisone acetate, oxytocin, vassopressin, |
| | glucagon and their derivatives |
| antiprotozoans | chloroquine, hydroxychloroquine, metronidazole, |
| | quinine and meglumine antimonite |
| antituberculars | para-aminosalicylic acid, isoniazid, capreomycin |
| | sulfate cycloserine, ethambutol hydrochloride |
| | ethionamide, pyrazinamide, rifam pin, and |
| | streptomycin sulfate |
| cardiovascular products | chelating agents and mercurial diuretics and cardiac |
| | glycosides |
| blood products | parenteral iron, hemin, hematoporphyrins and their |
| | derivatives |
| biological response | cytokines, muramyldipeptide, muramyltripeptide, |
| modifiers | microbial cell wall components, lymphokines (e.g., |
| | bacterial endotoxin such as lipopolysaccharide, |
| | macrophage activation factor), sub-units of bacteria |
| | (such as Mycobacteria, Corynebacteria), the |
| | synthetic dipeptide N-acetyl-muramyl-L-alanyl-D- |
| | isoglutamine |
| anti-fungal agents | ketoconazole, nystatin, griseofulvin, flucytosine (5- |
| | fc), miconazole, amphotericin B, ricin, cyclosporins, |
| | and β-lactam antibiotics (e.g., sulfazecin) |
| vitamins | cyanocobalamin neinoic acid, retinoids and |
| | derivatives such as retinol palm itate, and a- |
| | tocopherol |
| Peptides | manganese super oxide dismutase; enzymes such |
| | as alkaline phosphatase |
| anti-allergic agents | Amelexanox |
| anti-coagulation agents | phenprocoumon and heparin |
| circulatory drugs | Propranolol |
| metabolic potentiators | Glutathione |
| antivirals | acyclovir, amantadine azidothymidine (AZT, DDI, |
| | Foscarnet, or Zidovudine), ribavirin and vidarabine |
| | monohydrate (adenine arabinoside, ara-A) |
| antianginals | diltiazem, nifedipine, verapamil, erythritol tetranitrate, |
| | isosorbide dinitrate, nitroglycerin (glyceryl trinitrate) |
| | and pentaerythritol tetranitrate |
| antibiotics | dapsone, chloramphenicol, neomycin, cefaclor, |
| | cefadroxil, cephalexin, cephradine erythromycin, |
| | clindamycin, lincomycin, amoxicillin, ampicillin, |
| | bacampicillin, carbenicillin, dicloxacillin, cyclacillin, |
| | picloxacillin, hetacillin, methicillin, nafcillin, oxacillin, |
| | penicillin including penicillin G and penicillin V, |
| | ticarcillin rifampin, aminoglycosides and tetracycline |
| antiinflammatories | diflunisal, ibuprofen, indomethacin, meclofenamate, |
| | mefenamic acid, naproxen, oxyphenbutazone, |
| | phenylbutazone, piroxicam, sulindac, tolmetin, |
| | aspirin and salicylates |
| antirheumatics | Adalimumab, azathioprine, chloroquine and |
| | hydroxychloroquine (antimalarials), |
| | cyclosporine (Cyclosporin A), D-penicillamine, |
| | etanercept, gold salts (sodium aurothiomalate, |
| | auranofin), infliximab, leflunomide, methotrexate, |
| | minocycline (a tetracycline antibiotic), sulfasalazine |
| narcotics | Paregoric, opiates, codeine, heroin, methadone, |
| | morphine and opium |
| cardiac glycosides | deslanoside, digitoxin, digoxin, digitalin and digitalis |
| neuromuscular blockers | atracurium mesylate, gallamine triethiodide, |
| | hexafluorenium bromide, metocurine iodide, |
| | pancuronium bromide, succinylcholine chloride |
| | (suxamethonium chloride), tubocurarine chloride and |
| | vecuronium bromide |
| sedatives (hypnotics) | amobarbital, amobarbital sodium, aprobarbital, |
| | butabarbital sodium, chloral hydrate, ethchlorvynol, |
| | ethinamate, flurazepam hydrochloride, glutethimide, |
| | methotrimeprazine hydrochloride, methyprylon, |
| | midazolam hydrochloride, paraldehyde, |
| | pentobarbital, pentobarbital sodium, phenobarbital |
| | sodium, secobarbital sodium, talbutal, temazepam |
| | and triazolam |
| local anesthetics | bupivacaine hydrochloride, chloroprocaine |
| | hydrochloride, etidocaine hydrochloride, lidocaine |
| | hydrochloride, mepivacaine hydrochloride, procaine |
| | hydrochloride and tetracaine hydrochloride |
| general anesthetics | droperidol, etomidate, fentanyl citrate with droperidol, |
| | ketamine hydrochloride, methohexital sodium and |
| | thiopental sodium |
| radioactive particles or | Actinium, lead²¹², radium, strontium, iodide rhenium, |
| ions | yttrium, and radiopharmaceuticals, such as |
| | radioactive iodine, copper and phosphorus product |

One aspect of the present invention encompasses an antibody that binds to GRP78. In some embodiments, the antiGRP78 antibody is derived from a hybridoma designated 7A9 or 6F8. As used herein, the term “derived from” means that the “derived” antibody comprises at least one CDR region from the antibody produced by 6F8 or 7A9. Stated another way, the “derived antibody” comprises at least one amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:4, or SEQ ID NO:12.

In one embodiment, an antibody of the disclosure may be derived from the hybridoma 7A9, and may comprise an amino acid sequence with 90, 91, 92, 93, 94, 95, 96, 97, 98, or 99% identity to the heavy chain variable region of SEQ ID NO:13, and/or may comprise an amino acid sequence with 90, 91, 92, 93, 94, 95, 96, 97, 98, or 99% identity to the light chain variable region of SEQ ID NO:14. In another embodiment, an antibody of the disclosure may be derived from the hybridoma 6F8, and may comprise an amino acid sequence with 90, 91, 92, 93, 94, 95, 96, 97, 98, or 99% identity to the heavy chain variable region of SEQ ID NO:15, and/or may comprise an amino acid sequence with 90, 91, 92, 93, 94, 95, 96, 97, 98, or 99% identity to the light chain variable region of SEQ ID NO:16. In another embodiment, an antibody of the disclosure may comprise an amino acid sequence with 90, 91, 92, 93, 94, 95, 96, 97, 98, or 99% identity to the heavy chain variable region of SEQ ID NO:27, and/or may comprise an amino acid sequence with 90, 91, 92, 93, 94, 95, 96, 97, 98, or 99% identity to the light chain variable region of SEQ ID NO:28. In another embodiment, an antibody of the disclosure may comprise an amino acid sequence with 90, 91, 92, 93, 94, 95, 96, 97, 98, or 99% identity to the heavy chain variable region of SEQ ID NO:35, and/or may comprise an amino acid sequence with 90, 91, 92, 93, 94, 95, 96, 97, 98, or 99% identity to the light chain variable region of SEQ ID NO:36. In another embodiment, an antibody of the disclosure may comprise an amino acid sequence with 90, 91, 92, 93, 94, 95, 96, 97, 98, or 99% identity to the heavy chain variable region of SEQ ID NO:43, and/or may comprise an amino acid sequence with 90, 91, 92, 93, 94, 95, 96, 97, 98, or 99% identity to the light chain variable region of SEQ ID NO:44. In another embodiment, an antibody of the disclosure may comprise an amino acid sequence with 90, 91, 92, 93, 94, 95, 96, 97, 98, or 99% identity to the heavy chain variable region of SEQ ID NO:51, and/or may comprise an amino acid sequence with 90, 91, 92, 93, 94, 95, 96, 97, 98, or 99% identity to the light chain variable region of SEQ ID NO:52. In each of the above embodiments, the antibody may be humanized.

In an exemplary embodiment, an antibody of the disclosure that binds GRP78 comprises the heavy chain amino acid sequence of SEQ ID NO:13 and the light chain amino acid sequence of SEQ ID NO:14 [i.e. the monoclonal antibody referred to as 7A9]. In another exemplary embodiment, an antibody of the disclosure that binds to GRP78 comprises the heavy chain amino acid sequence of SEQ ID NO:15 and the light chain amino acid sequence of SEQ ID NO:16 [i.e. the monoclonal antibody referred to as 6F8]. In another exemplary embodiment, an antibody of the disclosure that binds to GRP78 comprises the heavy chain amino acid sequence of SEQ ID NO:27 and the light chain amino acid sequence of SEQ ID NO:28 [i.e. the scFV antibody referred to as GRP78-1183]. In another exemplary embodiment, an antibody of the disclosure that binds to GRP78 comprises the heavy chain amino acid sequence of SEQ ID NO:35 and the light chain amino acid sequence of SEQ ID NO:36 [i.e. the scFV antibody referred to as GRP78-1164]. In another exemplary embodiment, an antibody of the disclosure that binds to GRP78 comprises the heavy chain amino acid sequence of SEQ ID NO:43 and the light chain amino acid sequence of SEQ ID NO:44 [i.e. the scFV antibody referred to as GRP78-1171]. In another exemplary embodiment, an antibody of the disclosure that binds to GRP78 comprises the heavy chain amino acid sequence of SEQ ID NO:51 and the light chain amino acid sequence of SEQ ID NO:52 [i.e. the scFV antibody referred to as GRP78-1256].

In one embodiment, an antibody of the disclosure may comprise a heavy chain CDR1, such as antibody 1, 49, 97, 146, 194, and 242 of Table B. In another embodiment, an antibody of the disclosure may comprise a heavy chain CDR2, such as antibody 4, 52, 100, 149, 197 and 245 of Table B. In yet another embodiment, an antibody of the disclosure may comprise a heavy chain CDR3, such as antibody 6, 54, 102, 151, 196 and 247 of Table B. In an alternative embodiment, an antibody of the disclosure may comprise a combination of two or three heavy chain CDRs, such as the antibodies 2, 3, 5, 50, 51, 53, 98, 99, 101, 147, 148, 150, 195, 196 and 198 of Table B.

Similarly, in one embodiment, an antibody of the disclosure may comprise a light chain CDR1, such as antibody 7, 55, 103, 152, 200, and 248 of Table B. In another embodiment, an antibody of the disclosure may comprise a light chain CDR2, such as antibody 10, 58, 106, 155, 203 and 251 of Table B. In yet another embodiment, an antibody of the disclosure may comprise a light chain CDR3, such as antibody 12, 60, 108, 157, 205 and 253 of Table B. In an alternative embodiment, an antibody of the disclosure may comprise a combination of two or three light chain CDRs, such as the antibodies 8, 9, 11, 56, 57, 59, 104, 105, 107, 153, 154, 156, 201, 202, 204, 249, 250, and 252 of Table B.

Alternatively, an antibody of the disclosure may comprise one or more light chain CDRs and one or more heavy chain CDRs, such as the antibodies 13-48, 61-96, 109-145, 158-193, 200-226, and 233-286 of Table B.

In an exemplary embodiment, an antibody of the invention may comprise a combination of CDR sequences listed in Table B below.

TABLE B
CDR combinations comprising antibodies that recognize GRP78

| Anti- | Heavy Chain | | | Light Chain | | |
|---|---|---|---|---|---|---|
| body | CDR1 | CDR2 | CDR3 | CDR1 | CDR2 | CDR3 |
| 1 | SEQ ID NO: 1 | | | | | |
| 2 | SEQ ID NO: 1 | SEQ ID NO: 2 | | | | |
| 3 | SEQ ID NO: 1 | SEQ ID NO: 2 | SEQ ID NO: 3 | | | |
| 4 | | SEQ ID NO: 2 | | | | |
| 5 | | SEQ ID NO: 2 | SEQ ID NO: 3 | | | |
| 6 | | | SEQ ID NO: 3 | | | |
| 7 | | | | SEQ ID NO: 4 | | |
| 8 | | | | SEQ ID NO: 4 | SEQ ID NO: 5 | |
| 9 | | | | SEQ ID NO: 4 | SEQ ID NO: 5 | SEQ ID NO: 6 |
| 10 | | | | | SEQ ID NO: 5 | |
| 11 | | | | | SEQ ID NO: 5 | SEQ ID NO: 6 |
| 12 | | | | | | SEQ ID NO: 6 |
| 13 | SEQ ID NO: 1 | | | SEQ ID NO: 4 | | |
| 14 | SEQ ID NO: 1 | | | SEQ ID NO: 4 | SEQ ID NO: 5 | |
| 15 | SEQ ID NO: 1 | | | SEQ ID NO: 4 | SEQ ID NO: 5 | SEQ ID NO: 6 |
| 16 | SEQ ID NO: 1 | | | | SEQ ID NO: 5 | |
| 17 | SEQ ID NO: 1 | | | | SEQ ID NO: 5 | SEQ ID NO: 6 |
| 18 | SEQ ID NO: 1 | | | | | SEQ ID NO: 6 |
| 19 | SEQ ID NO: 1 | SEQ ID NO: 2 | | SEQ ID NO: 4 | | |
| 20 | SEQ ID NO: 1 | SEQ ID NO: 2 | | SEQ ID NO: 4 | SEQ ID NO: 5 | |
| 21 | SEQ ID NO: 1 | SEQ ID NO: 2 | | SEQ ID NO: 4 | SEQ ID NO: 5 | SEQ ID NO: 6 |
| 22 | SEQ ID NO: 1 | SEQ ID NO: 2 | | | SEQ ID NO: 5 | |
| 23 | SEQ ID NO: 1 | SEQ ID NO: 2 | | | SEQ ID NO: 5 | SEQ ID NO: 6 |
| 24 | SEQ ID NO: 1 | SEQ ID NO: 2 | | | | SEQ ID NO: 6 |
| 25 | SEQ ID NO: 1 | SEQ ID NO: 2 | SEQ ID NO: 3 | SEQ ID NO: 4 | | |
| 26 | SEQ ID NO: 1 | SEQ ID NO: 2 | SEQ ID NO: 3 | SEQ ID NO: 4 | SEQ ID NO: 5 | |
| 27 | SEQ ID NO: 1 | SEQ ID NO: 2 | SEQ ID NO: 3 | SEQ ID NO: 4 | SEQ ID NO: 5 | SEQ ID NO: 6 |
| 28 | SEQ ID NO: 1 | SEQ ID NO: 2 | SEQ ID NO: 3 | | SEQ ID NO: 5 | |
| 29 | SEQ ID NO: 1 | SEQ ID NO: 2 | SEQ ID NO: 3 | | SEQ ID NO: 5 | SEQ ID NO: 6 |
| 30 | SEQ ID NO: 1 | SEQ ID NO: 2 | SEQ ID NO: 3 | | | SEQ ID NO: 6 |
| 31 | | SEQ ID NO: 2 | | SEQ ID NO: 4 | | |
| 32 | | SEQ ID NO: 2 | | SEQ ID NO: 4 | SEQ ID NO: 5 | |
| 33 | | SEQ ID NO: 2 | | SEQ ID NO: 4 | SEQ ID NO: 5 | SEQ ID NO: 6 |
| 34 | | SEQ ID NO: 2 | | | SEQ ID NO: 5 | |
| 35 | | SEQ ID NO: 2 | | | SEQ ID NO: 5 | SEQ ID NO: 6 |
| 36 | | SEQ ID NO: 2 | | | | SEQ ID NO: 6 |
| 37 | | SEQ ID NO: 2 | SEQ ID NO: 3 | SEQ ID NO: 4 | | |
| 38 | | SEQ ID NO: 2 | SEQ ID NO: 3 | SEQ ID NO: 4 | SEQ ID NO: 5 | |
| 39 | | SEQ ID NO: 2 | SEQ ID NO: 3 | SEQ ID NO: 4 | SEQ ID NO: 5 | SEQ ID NO: 6 |
| 40 | | SEQ ID NO: 2 | SEQ ID NO: 3 | | SEQ ID NO: 5 | |
| 41 | | SEQ ID NO: 2 | SEQ ID NO: 3 | | SEQ ID NO: 5 | SEQ ID NO: 6 |
| 42 | | SEQ ID NO: 2 | SEQ ID NO: 3 | | | SEQ ID NO: 6 |
| 43 | | | SEQ ID NO: 3 | SEQ ID NO: 4 | | |
| 44 | | | SEQ ID NO: 3 | SEQ ID NO: 4 | SEQ ID NO: 5 | |
| 45 | | | SEQ ID NO: 3 | SEQ ID NO: 4 | SEQ ID NO: 5 | SEQ ID NO: 6 |
| 46 | | | SEQ ID NO: 3 | | SEQ ID NO: 5 | |
| 47 | | | SEQ ID NO: 3 | | SEQ ID NO: 5 | SEQ ID NO: 6 |
| 48 | | | SEQ ID NO: 3 | | | SEQ ID NO: 6 |
| 49 | SEQ ID NO: 7 | | | | | |
| 50 | SEQ ID NO: 7 | SEQ ID NO: 8 | | | | |
| 51 | SEQ ID NO: 7 | SEQ ID NO: 8 | SEQ ID NO: 9 | | | |
| 52 | | SEQ ID NO: 8 | | | | |
| 53 | | SEQ ID NO: 8 | SEQ ID NO: 9 | | | |
| 54 | | | SEQ ID NO: 9 | | | |
| 55 | | | | SEQ ID NO: 10 | | |
| 56 | | | | SEQ ID NO: 10 | SEQ ID NO: 11 | |
| 57 | | | | SEQ ID NO: 10 | SEQ ID NO: 11 | SEQ ID NO: 12 |
| 58 | | | | | SEQ ID NO: 11 | |
| 59 | | | | | SEQ ID NO: 11 | SEQ ID NO: 12 |
| 60 | | | | | | SEQ ID NO: 12 |
| 61 | SEQ ID NO: 7 | | | SEQ ID NO: 10 | | |
| 62 | SEQ ID NO: 7 | | | SEQ ID NO: 10 | SEQ ID NO: 11 | |
| 63 | SEQ ID NO: 7 | | | SEQ ID NO: 10 | SEQ ID NO: 11 | SEQ ID NO: 12 |
| 64 | SEQ ID NO: 7 | | | | SEQ ID NO: 11 | |
| 65 | SEQ ID NO: 7 | | | | SEQ ID NO: 11 | SEQ ID NO: 12 |
| 66 | SEQ ID NO: 7 | | | | | SEQ ID NO: 12 |
| 67 | SEQ ID NO: 7 | SEQ ID NO: 8 | | SEQ ID NO: 10 | | |
| 68 | SEQ ID NO: 7 | SEQ ID NO: 8 | | SEQ ID NO: 10 | SEQ ID NO: 11 | |
| 69 | SEQ ID NO: 7 | SEQ ID NO: 8 | | SEQ ID NO: 10 | SEQ ID NO: 11 | SEQ ID NO: 12 |
| 70 | SEQ ID NO: 7 | SEQ ID NO: 8 | | | SEQ ID NO: 11 | |
| 71 | SEQ ID NO: 7 | SEQ ID NO: 8 | | | SEQ ID NO: 11 | SEQ ID NO: 12 |
| 72 | SEQ ID NO: 7 | SEQ ID NO: 8 | | | | SEQ ID NO: 12 |
| 73 | SEQ ID NO: 7 | SEQ ID NO: 8 | SEQ ID NO: 9 | SEQ ID NO: 10 | | |
| 74 | SEQ ID NO: 7 | SEQ ID NO: 8 | SEQ ID NO: 9 | SEQ ID NO: 10 | SEQ ID NO: 11 | |
| 75 | SEQ ID NO: 7 | SEQ ID NO: 8 | SEQ ID NO: 9 | SEQ ID NO: 10 | SEQ ID NO: 11 | SEQ ID NO: 12 |
| 76 | SEQ ID NO: 7 | SEQ ID NO: 8 | SEQ ID NO: 9 | | SEQ ID NO: 11 | |
| 77 | SEQ ID NO: 7 | SEQ ID NO: 8 | SEQ ID NO: 9 | | SEQ ID NO: 11 | SEQ ID NO: 12 |
| 78 | SEQ ID NO: 7 | SEQ ID NO: 8 | SEQ ID NO: 9 | | | SEQ ID NO: 12 |
| 79 | | SEQ ID NO: 8 | | SEQ ID NO: 10 | | |
| 80 | | SEQ ID NO: 8 | | SEQ ID NO: 10 | SEQ ID NO: 11 | |
| 81 | | SEQ ID NO: 8 | | SEQ ID NO: 10 | SEQ ID NO: 11 | SEQ ID NO: 12 |
| 82 | | SEQ ID NO: 8 | | | SEQ ID NO: 11 | |
| 83 | | SEQ ID NO: 8 | | | SEQ ID NO: 11 | SEQ ID NO: 12 |
| 84 | | SEQ ID NO: 8 | | | | SEQ ID NO: 12 |
| 85 | | SEQ ID NO: 8 | SEQ ID NO: 9 | SEQ ID NO: 10 | | |
| 86 | | SEQ ID NO: 8 | SEQ ID NO: 9 | SEQ ID NO: 10 | SEQ ID NO: 11 | |
| 87 | | SEQ ID NO: 8 | SEQ ID NO: 9 | SEQ ID NO: 10 | SEQ ID NO: 11 | SEQ ID NO: 12 |
| 88 | | SEQ ID NO: 8 | SEQ ID NO: 9 | | SEQ ID NO: 11 | |
| 89 | | SEQ ID NO: 8 | SEQ ID NO: 9 | | SEQ ID NO: 11 | SEQ ID NO: 12 |
| 90 | | SEQ ID NO: 8 | SEQ ID NO: 9 | | | SEQ ID NO: 12 |
| 91 | | | SEQ ID NO: 9 | SEQ ID NO: 10 | | |
| 92 | | | SEQ ID NO: 9 | SEQ ID NO: 10 | SEQ ID NO: 11 | |
| 93 | | | SEQ ID NO: 9 | SEQ ID NO: 10 | SEQ ID NO: 11 | SEQ ID NO: 12 |
| 94 | | | SEQ ID NO: 9 | | SEQ ID NO: 11 | |
| 95 | | | SEQ ID NO: 9 | | SEQ ID NO: 11 | SEQ ID NO: 12 |
| 96 | | | SEQ ID NO: 9 | | | SEQ ID NO: 12 |
| 97 | SEQ ID NO: 21 | | | | | |
| 98 | SEQ ID NO: 21 | SEQ ID NO: 22 | | | | |
| 99 | SEQ ID NO: 21 | SEQ ID NO: 22 | SEQ ID NO: 23 | | | |
| 100 | | SEQ ID NO: 22 | | | | |
| 101 | | SEQ ID NO: 22 | SEQ ID NO: 23 | | | |
| 102 | | | SEQ ID NO: 23 | | | |
| 103 | | | | SEQ ID NO: 24 | | |
| 104 | | | | SEQ ID NO: 24 | SEQ ID NO: 25 | |
| 105 | | | | SEQ ID NO: 24 | SEQ ID NO: 25 | SEQ ID NO: 26 |
| 106 | | | | | SEQ ID NO: 25 | |
| 107 | | | | | SEQ ID NO: 25 | SEQ ID NO: 26 |
| 108 | | | | | | SEQ ID NO: 26 |
| 109 | SEQ ID NO: 21 | | | SEQ ID NO: 24 | | |
| 110 | SEQ ID NO: 21 | | | SEQ ID NO: 24 | SEQ ID NO: 25 | |
| 111 | SEQ ID NO: 21 | | | SEQ ID NO: 24 | SEQ ID NO: 25 | SEQ ID NO: 26 |
| 112 | SEQ ID NO: 21 | | | | SEQ ID NO: 25 | |
| 113 | SEQ ID NO: 21 | | | | SEQ ID NO: 25 | SEQ ID NO: 26 |
| 114 | SEQ ID NO: 21 | | | | | SEQ ID NO: 26 |
| 115 | SEQ ID NO: 21 | SEQ ID NO: 22 | | SEQ ID NO: 24 | | |
| 116 | SEQ ID NO: 21 | SEQ ID NO: 22 | | SEQ ID NO: 24 | SEQ ID NO: 25 | |
| 117 | SEQ ID NO: 21 | SEQ ID NO: 22 | | SEQ ID NO: 24 | SEQ ID NO: 25 | SEQ ID NO: 26 |
| 118 | SEQ ID NO: 21 | SEQ ID NO: 22 | | | SEQ ID NO: 25 | |
| 119 | SEQ ID NO: 21 | SEQ ID NO: 22 | | | SEQ ID NO: 25 | SEQ ID NO: 26 |
| 120 | SEQ ID NO: 21 | SEQ ID NO: 22 | | | | SEQ ID NO: 26 |
| 121 | SEQ ID NO: 21 | SEQ ID NO: 22 | SEQ ID NO: 23 | SEQ ID NO: 24 | | |
| 123 | SEQ ID NO: 21 | SEQ ID NO: 22 | SEQ ID NO: 23 | SEQ ID NO: 24 | SEQ ID NO: 25 | |
| 124 | SEQ ID NO: 21 | SEQ ID NO: 22 | SEQ ID NO: 23 | SEQ ID NO: 24 | SEQ ID NO: 25 | SEQ ID NO: 26 |
| 125 | SEQ ID NO: 21 | SEQ ID NO: 22 | SEQ ID NO: 23 | | SEQ ID NO: 25 | |
| 126 | SEQ ID NO: 21 | SEQ ID NO: 22 | SEQ ID NO: 23 | | SEQ ID NO: 25 | SEQ ID NO: 26 |
| 127 | SEQ ID NO: 21 | SEQ ID NO: 22 | SEQ ID NO: 23 | | | SEQ ID NO: 26 |
| 128 | | SEQ ID NO: 22 | | SEQ ID NO: 24 | | |
| 129 | | SEQ ID NO: 22 | | SEQ ID NO: 24 | SEQ ID NO: 25 | |
| 130 | | SEQ ID NO: 22 | | SEQ ID NO: 24 | SEQ ID NO: 25 | SEQ ID NO: 26 |
| 131 | | SEQ ID NO: 22 | | | SEQ ID NO: 25 | |
| 132 | | SEQ ID NO: 22 | | | SEQ ID NO: 25 | SEQ ID NO: 26 |
| 133 | | SEQ ID NO: 22 | | | | SEQ ID NO: 26 |
| 134 | | SEQ ID NO: 22 | SEQ ID NO: 23 | SEQ ID NO: 24 | | |
| 135 | | SEQ ID NO: 22 | SEQ ID NO: 23 | SEQ ID NO: 24 | SEQ ID NO: 25 | |
| 136 | | SEQ ID NO: 22 | SEQ ID NO: 23 | SEQ ID NO: 24 | SEQ ID NO: 25 | SEQ ID NO: 26 |
| 137 | | SEQ ID NO: 22 | SEQ ID NO: 23 | | SEQ ID NO: 25 | |
| 138 | | SEQ ID NO: 22 | SEQ ID NO: 23 | | SEQ ID NO: 25 | SEQ ID NO: 26 |
| 139 | | SEQ ID NO: 22 | SEQ ID NO: 23 | | | SEQ ID NO: 26 |
| 140 | | | SEQ ID NO: 23 | SEQ ID NO: 24 | | |
| 141 | | | SEQ ID NO: 23 | SEQ ID NO: 24 | SEQ ID NO: 25 | |
| 142 | | | SEQ ID NO: 23 | SEQ ID NO: 24 | SEQ ID NO: 25 | SEQ ID NO: 26 |
| 143 | | | SEQ ID NO: 23 | | SEQ ID NO: 25 | |
| 144 | | | SEQ ID NO: 23 | | SEQ ID NO: 25 | SEQ ID NO: 26 |
| 145 | | | SEQ ID NO: 23 | | | SEQ ID NO: 26 |
| 146 | SEQ ID NO: 29 | | | | | |
| 147 | SEQ ID NO: 29 | SEQ ID NO: 30 | | | | |
| 148 | SEQ ID NO: 29 | SEQ ID NO: 30 | SEQ ID NO: 31 | | | |
| 149 | | SEQ ID NO: 30 | | | | |
| 150 | | SEQ ID NO: 30 | SEQ ID NO: 31 | | | |
| 151 | | | SEQ ID NO: 31 | | | |
| 152 | | | | SEQ ID NO: 32 | | |
| 153 | | | | SEQ ID NO: 32 | SEQ ID NO: 33 | |
| 154 | | | | SEQ ID NO: 32 | SEQ ID NO: 33 | SEQ ID NO: 34 |
| 155 | | | | | SEQ ID NO: 33 | |
| 156 | | | | | SEQ ID NO: 33 | SEQ ID NO: 34 |
| 157 | | | | | | SEQ ID NO: 34 |
| 158 | SEQ ID NO: 29 | | | SEQ ID NO: 32 | | |
| 159 | SEQ ID NO: 29 | | | SEQ ID NO: 32 | SEQ ID NO: 33 | |
| 160 | SEQ ID NO: 29 | | | SEQ ID NO: 32 | SEQ ID NO: 33 | SEQ ID NO: 34 |
| 161 | SEQ ID NO: 29 | | | | SEQ ID NO: 33 | |
| 162 | SEQ ID NO: 29 | | | | SEQ ID NO: 33 | SEQ ID NO: 34 |
| 163 | SEQ ID NO: 29 | | | | | SEQ ID NO: 34 |
| 164 | SEQ ID NO: 29 | SEQ ID NO: 30 | | SEQ ID NO: 32 | | |
| 165 | SEQ ID NO: 29 | SEQ ID NO: 30 | | SEQ ID NO: 32 | SEQ ID NO: 33 | |
| 166 | SEQ ID NO: 29 | SEQ ID NO: 30 | | SEQ ID NO: 32 | SEQ ID NO: 33 | SEQ ID NO: 34 |
| 167 | SEQ ID NO: 29 | SEQ ID NO: 30 | | | SEQ ID NO: 33 | |
| 168 | SEQ ID NO: 29 | SEQ ID NO: 30 | | | SEQ ID NO: 33 | SEQ ID NO: 34 |
| 169 | SEQ ID NO: 29 | SEQ ID NO: 30 | | | | SEQ ID NO: 34 |
| 170 | SEQ ID NO: 29 | SEQ ID NO: 30 | SEQ ID NO: 31 | SEQ ID NO: 32 | | |
| 171 | SEQ ID NO: 29 | SEQ ID NO: 30 | SEQ ID NO: 31 | SEQ ID NO: 32 | SEQ ID NO: 33 | |
| 172 | SEQ ID NO: 29 | SEQ ID NO: 30 | SEQ ID NO: 31 | SEQ ID NO: 32 | SEQ ID NO: 33 | SEQ ID NO: 34 |
| 173 | SEQ ID NO: 29 | SEQ ID NO: 30 | SEQ ID NO: 31 | | SEQ ID NO: 33 | |
| 174 | SEQ ID NO: 29 | SEQ ID NO: 30 | SEQ ID NO: 31 | | SEQ ID NO: 33 | SEQ ID NO: 34 |
| 175 | SEQ ID NO: 29 | SEQ ID NO: 30 | SEQ ID NO: 31 | | | SEQ ID NO: 34 |
| 176 | | SEQ ID NO: 30 | | SEQ ID NO: 32 | | |
| 177 | | SEQ ID NO: 30 | | SEQ ID NO: 32 | SEQ ID NO: 33 | |
| 178 | | SEQ ID NO: 30 | | SEQ ID NO: 32 | SEQ ID NO: 33 | SEQ ID NO: 34 |
| 179 | | SEQ ID NO: 30 | | | SEQ ID NO: 33 | |
| 180 | | SEQ ID NO: 30 | | | SEQ ID NO: 33 | SEQ ID NO: 34 |
| 181 | | SEQ ID NO: 30 | | | | SEQ ID NO: 34 |
| 182 | | SEQ ID NO: 30 | SEQ ID NO: 31 | SEQ ID NO: 32 | | |
| 183 | | SEQ ID NO: 30 | SEQ ID NO: 31 | SEQ ID NO: 32 | SEQ ID NO: 33 | |
| 184 | | SEQ ID NO: 30 | SEQ ID NO: 31 | SEQ ID NO: 32 | SEQ ID NO: 33 | SEQ ID NO: 34 |
| 185 | | SEQ ID NO: 30 | SEQ ID NO: 31 | | SEQ ID NO: 33 | |
| 186 | | SEQ ID NO: 30 | SEQ ID NO: 31 | | SEQ ID NO: 33 | SEQ ID NO: 34 |
| 187 | | SEQ ID NO: 30 | SEQ ID NO: 31 | | | SEQ ID NO: 34 |
| 188 | | | SEQ ID NO: 31 | SEQ ID NO: 32 | | |
| 189 | | | SEQ ID NO: 31 | SEQ ID NO: 32 | SEQ ID NO: 33 | |
| 190 | | | SEQ ID NO: 31 | SEQ ID NO: 32 | SEQ ID NO: 33 | SEQ ID NO: 34 |
| 191 | | | SEQ ID NO: 31 | | SEQ ID NO: 33 | |
| 192 | | | SEQ ID NO: 31 | | SEQ ID NO: 33 | SEQ ID NO: 34 |
| 193 | | | SEQ ID NO: 31 | | | SEQ ID NO: 34 |
| 194 | SEQ ID NO: 37 | | | | | |
| 195 | SEQ ID NO: 37 | SEQ ID NO: 38 | | | | |
| 196 | SEQ ID NO: 37 | SEQ ID NO: 38 | SEQ ID NO: 39 | | | |
| 197 | | SEQ ID NO: 38 | | | | |
| 198 | | SEQ ID NO: 38 | SEQ ID NO: 39 | | | |
| 199 | | | SEQ ID NO: 39 | | | |
| 200 | | | | SEQ ID NO: 40 | | |
| 201 | | | | SEQ ID NO: 40 | SEQ ID NO: 41 | |
| 202 | | | | SEQ ID NO: 40 | SEQ ID NO: 41 | SEQ ID NO: 42 |
| 203 | | | | | SEQ ID NO: 41 | |
| 204 | | | | | SEQ ID NO: 41 | SEQ ID NO: 42 |
| 205 | | | | | | SEQ ID NO: 42 |
| 206 | SEQ ID NO: 37 | | | SEQ ID NO: 40 | | |
| 207 | SEQ ID NO: 37 | | | SEQ ID NO: 40 | SEQ ID NO: 41 | |
| 208 | SEQ ID NO: 37 | | | SEQ ID NO: 40 | SEQ ID NO: 41 | SEQ ID NO: 42 |
| 209 | SEQ ID NO: 37 | | | | SEQ ID NO: 41 | |
| 210 | SEQ ID NO: 37 | | | | SEQ ID NO: 41 | SEQ ID NO: 42 |
| 211 | SEQ ID NO: 37 | | | | | SEQ ID NO: 42 |
| 212 | SEQ ID NO: 37 | SEQ ID NO: 38 | | SEQ ID NO: 40 | | |
| 213 | SEQ ID NO: 37 | SEQ ID NO: 38 | | SEQ ID NO: 40 | SEQ ID NO: 41 | |
| 214 | SEQ ID NO: 37 | SEQ ID NO: 38 | | SEQ ID NO: 40 | SEQ ID NO: 41 | SEQ ID NO: 42 |
| 215 | SEQ ID NO: 37 | SEQ ID NO: 38 | | | SEQ ID NO: 41 | |
| 216 | SEQ ID NO: 37 | SEQ ID NO: 38 | | | SEQ ID NO: 41 | SEQ ID NO: 42 |
| 217 | SEQ ID NO: 37 | SEQ ID NO: 38 | | | | SEQ ID NO: 42 |
| 218 | SEQ ID NO: 37 | SEQ ID NO: 38 | SEQ ID NO: 39 | SEQ ID NO: 40 | | |
| 219 | SEQ ID NO: 37 | SEQ ID NO: 38 | SEQ ID NO: 39 | SEQ ID NO: 40 | SEQ ID NO: 41 | |
| 220 | SEQ ID NO: 37 | SEQ ID NO: 38 | SEQ ID NO: 39 | SEQ ID NO: 40 | SEQ ID NO: 41 | SEQ ID NO: 42 |
| 221 | SEQ ID NO: 37 | SEQ ID NO: 38 | SEQ ID NO: 39 | | SEQ ID NO: 41 | |
| 222 | SEQ ID NO: 37 | SEQ ID NO: 38 | SEQ ID NO: 39 | | SEQ ID NO: 41 | SEQ ID NO: 42 |
| 223 | SEQ ID NO: 37 | SEQ ID NO: 38 | SEQ ID NO: 39 | | | SEQ ID NO: 42 |
| 224 | | SEQ ID NO: 38 | | SEQ ID NO: 40 | | |
| 225 | | SEQ ID NO: 38 | | SEQ ID NO: 40 | SEQ ID NO: 41 | |
| 226 | | SEQ ID NO: 38 | | SEQ ID NO: 40 | SEQ ID NO: 41 | SEQ ID NO: 42 |
| 227 | | SEQ ID NO: 38 | | | SEQ ID NO: 41 | |
| 228 | | SEQ ID NO: 38 | | | SEQ ID NO: 41 | SEQ ID NO: 42 |
| 229 | | SEQ ID NO: 38 | | | | SEQ ID NO: 42 |
| 230 | | SEQ ID NO: 38 | SEQ ID NO: 39 | SEQ ID NO: 40 | | |
| 231 | | SEQ ID NO: 38 | SEQ ID NO: 39 | SEQ ID NO: 40 | SEQ ID NO: 41 | |
| 232 | | SEQ ID NO: 38 | SEQ ID NO: 39 | SEQ ID NO: 40 | SEQ ID NO: 41 | SEQ ID NO: 42 |
| 233 | | SEQ ID NO: 38 | SEQ ID NO: 39 | | SEQ ID NO: 41 | |
| 234 | | SEQ ID NO: 38 | SEQ ID NO: 39 | | SEQ ID NO: 41 | SEQ ID NO: 42 |
| 235 | | SEQ ID NO: 38 | SEQ ID NO: 39 | | | SEQ ID NO: 42 |
| 236 | | | SEQ ID NO: 39 | SEQ ID NO: 40 | | |
| 237 | | | SEQ ID NO: 39 | SEQ ID NO: 40 | SEQ ID NO: 41 | |
| 238 | | | SEQ ID NO: 39 | SEQ ID NO: 40 | SEQ ID NO: 41 | SEQ ID NO: 42 |
| 239 | | | SEQ ID NO: 39 | | SEQ ID NO: 41 | |
| 240 | | | SEQ ID NO: 39 | | SEQ ID NO: 41 | SEQ ID NO: 42 |
| 241 | | | SEQ ID NO: 39 | | | SEQ ID NO: 42 |
| 242 | SEQ ID NO: 45 | | | | | |
| 243 | SEQ ID NO: 45 | SEQ ID NO: 46 | | | | |
| 244 | SEQ ID NO: 45 | SEQ ID NO: 46 | SEQ ID NO: 47 | | | |
| 245 | | SEQ ID NO: 46 | | | | |
| 246 | | SEQ ID NO: 46 | SEQ ID NO: 47 | | | |
| 247 | | | SEQ ID NO: 47 | | | |
| 248 | | | | SEQ ID NO: 48 | | |
| 249 | | | | SEQ ID NO: 48 | SEQ ID NO: 49 | |
| 250 | | | | SEQ ID NO: 48 | SEQ ID NO: 49 | SEQ ID NO: 50 |
| 251 | | | | | SEQ ID NO: 49 | |
| 252 | | | | | SEQ ID NO: 49 | SEQ ID NO: 50 |
| 253 | | | | | | SEQ ID NO: 50 |
| 254 | SEQ ID NO: 45 | | | SEQ ID NO: 48 | | |
| 255 | SEQ ID NO: 45 | | | SEQ ID NO: 48 | SEQ ID NO: 49 | |
| 256 | SEQ ID NO: 45 | | | SEQ ID NO: 48 | SEQ ID NO: 49 | SEQ ID NO: 50 |
| 257 | SEQ ID NO: 45 | | | | SEQ ID NO: 49 | |
| 258 | SEQ ID NO: 45 | | | | SEQ ID NO: 49 | SEQ ID NO: 50 |
| 259 | SEQ ID NO: 45 | | | | | SEQ ID NO: 50 |
| 260 | SEQ ID NO: 45 | SEQ ID NO: 46 | | SEQ ID NO: 48 | | |
| 261 | SEQ ID NO: 45 | SEQ ID NO: 46 | | SEQ ID NO: 48 | SEQ ID NO: 49 | |
| 262 | SEQ ID NO: 45 | SEQ ID NO: 46 | | SEQ ID NO: 48 | SEQ ID NO: 49 | SEQ ID NO: 50 |
| 263 | SEQ ID NO: 45 | SEQ ID NO: 46 | | | SEQ ID NO: 49 | |
| 264 | SEQ ID NO: 45 | SEQ ID NO: 46 | | | SEQ ID NO: 49 | SEQ ID NO: 50 |
| 265 | SEQ ID NO: 45 | SEQ ID NO: 46 | | | | SEQ ID NO: 50 |
| 266 | SEQ ID NO: 45 | SEQ ID NO: 46 | | SEQ ID NO: 48 | SEQ ID NO: 47 | |
| 267 | SEQ ID NO: 45 | SEQ ID NO: 46 | SEQ ID NO: 47 | SEQ ID NO: 48 | SEQ ID NO: 49 | |
| 268 | SEQ ID NO: 45 | SEQ ID NO: 46 | SEQ ID NO: 47 | SEQ ID NO: 48 | SEQ ID NO: 49 | SEQ ID NO: 50 |
| 269 | SEQ ID NO: 45 | SEQ ID NO: 46 | SEQ ID NO: 47 | | SEQ ID NO: 49 | |
| 270 | SEQ ID NO: 45 | SEQ ID NO: 46 | SEQ ID NO: 47 | | SEQ ID NO: 49 | SEQ ID NO: 50 |
| 271 | SEQ ID NO: 45 | SEQ ID NO: 46 | SEQ ID NO: 47 | | | SEQ ID NO: 50 |
| 272 | | SEQ ID NO: 46 | | SEQ ID NO: 48 | | |
| 273 | | SEQ ID NO: 46 | | SEQ ID NO: 48 | SEQ ID NO: 49 | |
| 274 | | SEQ ID NO: 46 | | SEQ ID NO: 48 | SEQ ID NO: 49 | SEQ ID NO: 50 |
| 275 | | SEQ ID NO: 46 | | | SEQ ID NO: 49 | |
| 276 | | SEQ ID NO: 46 | | | SEQ ID NO: 49 | SEQ ID NO: 50 |
| 277 | | SEQ ID NO: 46 | | | | SEQ ID NO: 50 |
| 278 | | SEQ ID NO: 46 | SEQ ID NO: 47 | SEQ ID NO: 48 | | |
| 279 | | SEQ ID NO: 46 | SEQ ID NO: 47 | SEQ ID NO: 48 | SEQ ID NO: 49 | |
| 280 | | SEQ ID NO: 46 | SEQ ID NO: 47 | SEQ ID NO: 48 | SEQ ID NO: 49 | SEQ ID NO: 50 |
| 281 | | SEQ ID NO: 46 | SEQ ID NO: 47 | | SEQ ID NO: 49 | |
| 282 | | SEQ ID NO: 46 | SEQ ID NO: 47 | | SEQ ID NO: 49 | SEQ ID NO: 50 |
| 283 | | SEQ ID NO: 46 | SEQ ID NO: 47 | | | SEQ ID NO: 50 |
| 284 | | | SEQ ID NO: 47 | SEQ ID NO: 48 | | |
| 285 | | | SEQ ID NO: 47 | SEQ ID NO: 48 | SEQ ID NO: 49 | |
| 286 | | | SEQ ID NO: 47 | SEQ ID NO: 48 | SEQ ID NO: 49 | SEQ ID NO: 50 |

In some embodiments, an antigen binding peptide according to the disclosure comprises a heavy chain CDR1 of amino acid sequence SEQ ID NO:72; X1-X2-A-M-N, wherein X1 is a hydrophobic or polar neutral side chain amino acid and X2 is a polar neutral side chain amino acid. In some embodiments, X1 is a hydrophobic aliphatic or a polar neutral side chain.

In some embodiments, an antigen binding peptide according to the disclosure comprises a heavy chain CDR2 of amino acid sequence SEQ ID NO:73; R-I-R-S-K-X1-X2-N-Y-A-T-Y-Y-A-D-S-N-K-D, wherein X1 is a polar neutral side chain amino acid; and X2 is a hydrophobic or polar neutral side chain amino acid. In some embodiments, X2 a hydrophobic aromatic or a polar neutral side chain amino acid.

In some embodiments, an antigen binding peptide according to the disclosure comprises a light chain CDR1 of amino acid sequence SEQ ID NO:74; x-x-X1-S-X2-x-I-X3-x-H-x-x, wherein x is any amino acid; X1 is a polar neutral side chain amino acid; X2 is a polar neutral side chain amino acid; and X3 is a hydrophobic or polar neutral side chain amino acid. In some embodiments, x is selected from S, M, H, L, R, or A. In some embodiments, x3 is a hydrophobic aromatic or a polar neutral side chain amino acid.

In some embodiments, an antigen binding peptide according to the disclosure comprises a light chain CDR2 of amino acid sequence SEQ ID NO:75; X1-X2-S-X3-L-x-S, wherein x is any amino acid; X1 is a hydrophobic or polar neutral side chain amino acid; X2 is a hydrophobic or polar neutral side chain amino acid; and X3 is a polar neutral side chain amino acid. In some embodiments, x is selected from A or D. In some embodiments, X1 is a hydrophobic aliphatic or a polar neutral side chain amino acid. In some embodiments, X2 is a hydrophobic aliphatic or a polar neutral side chain amino acid.

In some embodiments, an antigen binding peptide according to the disclosure comprises a light chain CDR3 of amino acid sequence SEQ ID NO:76; X1-Q-X2-S-S-Y-P-X3-T, wherein x is any amino acid; X1 is a hydrophobic or polar neutral side chain amino acid; and X3 is a polar neutral side chain amino acid. In some embodiments, X2 is selected from Y or R. In some embodiments, X1 is a hydrophobic aliphatic or a polar neutral side chain amino acid.

In one embodiment, an antigen binding peptide of the disclosure may comprise a heavy chain variable region comprising SEQ ID NO:1 with zero to two amino acid substitutions, SEQ ID NO:2 with zero to two amino acid substitutions, and SEQ ID NO:3 with zero to two amino acid substitutions, and/or may comprise a light chain variable region comprising SEQ ID NO:4 with zero to two amino acid substitutions, SEQ ID NO:5 with zero to two amino acid substitutions, and SEQ ID NO:6 with zero to two amino acid substitutions. In another embodiment, an antigen binding peptide of the disclosure may comprise a heavy chain variable region SEQ ID NO:7 with zero to two amino acid substitutions, SEQ ID NO:8 with zero to two amino acid substitutions, SEQ ID NO:9 with zero to two amino acid substitutions, a light chain variable region comprising SEQ ID NO:10 with zero to two amino acid substitutions, SEQ ID NO:11 with zero to two amino acid substitutions, and SEQ ID NO:12 with zero to two amino acid substitutions. In one embodiment, an antigen binding peptide of the disclosure may comprise a heavy chain variable region comprising SEQ ID NO:21 with zero to two amino acid substitutions, SEQ ID NO:22 with zero to two amino acid substitutions, and SEQ ID NO:23 with zero to two amino acid substitutions, and/or may comprise a light chain variable region comprising SEQ ID NO:24 with zero to two amino acid substitutions, SEQ ID NO:25 with zero to two amino acid substitutions, and SEQ ID NO:26 with zero to two amino acid substitutions. In another embodiment, an antigen binding peptide of the disclosure may comprise a heavy chain variable region SEQ ID NO:29 with zero to two amino acid substitutions, SEQ ID NO:30 with zero to two amino acid substitutions, SEQ ID NO:31 with zero to two amino acid substitutions, a light chain variable region comprising SEQ ID NO:32 with zero to two amino acid substitutions, SEQ ID NO:33 with zero to two amino acid substitutions, and SEQ ID NO:34 with zero to two amino acid substitutions. In one embodiment, an antigen binding peptide of the disclosure may comprise a heavy chain variable region comprising SEQ ID NO:37 with zero to two amino acid substitutions, SEQ ID NO:38 with zero to two amino acid substitutions, and SEQ ID NO:39 with zero to two amino acid substitutions, and/or may comprise a light chain variable region comprising SEQ ID NO:40 with zero to two amino acid substitutions, SEQ ID NO:41 with zero to two amino acid substitutions, and SEQ ID NO:42 with zero to two amino acid substitutions. In another embodiment, an antigen binding peptide of the disclosure may comprise a heavy chain variable region SEQ ID NO:45 with zero to two amino acid substitutions, SEQ ID NO:46 with zero to two amino acid substitutions, SEQ ID NO:47 with zero to two amino acid substitutions, a light chain variable region comprising SEQ ID NO:48 with zero to two amino acid substitutions, SEQ ID NO:49 with zero to two amino acid substitutions, and SEQ ID NO:50 with zero to two amino acid substitutions.

In an exemplary embodiment, an antigen binding peptide of the disclosure may comprise a heavy chain variable region comprising SEQ ID NO:1, SEQ ID NO:2, SEQ ID NO:3, and a light chain variable region comprising SEQ ID NO:4, SEQ ID NO:5, and SEQ ID NO:6. In an exemplary embodiment, an antigen binding peptide of the disclosure may comprise a heavy chain variable region comprising SEQ ID NO:7, SEQ ID NO:8, SEQ ID NO:9, and a light chain variable region comprising SEQ ID NO:10, SEQ ID NO:11, and SEQ ID NO:12. In an exemplary embodiment, an antigen binding peptide of the disclosure may comprise a heavy chain variable region comprising SEQ ID NO:21, SEQ ID NO:22, SEQ ID NO:23, and a light chain variable region comprising SEQ ID NO:24, SEQ ID NO:25, and SEQ ID NO:26. In an exemplary embodiment, an antigen binding peptide of the disclosure may comprise a heavy chain variable region comprising SEQ ID NO:29, SEQ ID NO:30, SEQ ID NO:31, and a light chain variable region comprising SEQ ID NO:32, SEQ ID NO:33, and SEQ ID NO:34. In an exemplary embodiment, an antigen binding peptide of the disclosure may comprise a heavy chain variable region comprising SEQ ID NO:37, SEQ ID NO:38, SEQ ID NO:39, and a light chain variable region comprising SEQ ID NO:40, SEQ ID NO:41, and SEQ ID NO:42. In an exemplary embodiment, an antigen binding peptide of the disclosure may comprise a heavy chain variable region comprising SEQ ID NO:45, SEQ ID NO:46, SEQ ID NO:47, and a light chain variable region comprising SEQ ID NO:48, SEQ ID NO:49, and SEQ ID NO:50.

In some embodiments, the disclosure encompasses nucleic acids of SEQ ID NO: 17, 18, 19, 20, 55, 56, 59, or 60. The disclosure also encompasses the corresponding nucleic acid sequences which encode SEQ ID NO:1, SEQ ID NO:2, SEQ ID NO:3, SEQ ID NO:4, SEQ ID NO:5, and SEQ ID NO:6, SEQ ID NO:7, SEQ ID NO:8, SEQ ID NO:9, SEQ ID NO:10, SEQ ID NO:11, SEQ ID NO:12, SEQ ID NO:21, SEQ ID NO:22, SEQ ID NO:23, SEQ ID NO:24, SEQ ID NO:25, SEQ ID NO:26, SEQ ID NO:29, SEQ ID NO:30, SEQ ID NO:31, SEQ ID NO:32, SEQ ID NO:33, SEQ ID NO:34, SEQ ID NO:37, SEQ ID NO:38, SEQ ID NO:39, SEQ ID NO:40, SEQ ID NO:41, SEQ ID NO:42, SEQ ID NO:45, SEQ ID NO:46, SEQ ID NO:47, SEQ ID NO:48, SEQ ID NO:49, or SEQ ID NO:50, which can readily be determined by one of skill in the art, and may be incorporated into a vector or other large DNA molecule, such as a chromosome, in order to express an antibody of the disclosure.

The disclosure also encompasses a vector comprising a nucleic acid sequence capable of encoding an antibody of the disclosure. As used herein, a “vector” is defined as a nucleic acid molecule used as a vehicle to transfer genetic material. Vectors include but are not limited to, plasmids, phasmids, cosmids, transposable elements, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors. An expression vector encoding an antibody of the disclosure may be delivered to the cell using a viral vector or via a non-viral method of transfer. Viral vectors suitable for introducing nucleic acids into cells include retroviruses, adenoviruses, adeno-associated viruses, rhabdoviruses, and herpes viruses. Non-viral methods of nucleic acid transfer include naked nucleic acid, liposomes, and protein/nucleic acid conjugates. An expression construct encoding an antibody of the disclosure that is introduced to the cell may be linear or circular, may be single-stranded or double-stranded, and may be DNA, RNA, or any modification or combination thereof. The disclosure also encompasses a cell line comprising a vector comprising a nucleic acid sequence capable of encoding an antibody of the disclosure, in non-limiting examples, CAR T-cells, CAR immune effector cells, and CAR macrophages. In some embodiments, the antigen binding peptides as disclosed herein are used to target a therapeutic virus (e.g. an engineered adenovirus, oncolytic virus) to a tumor cell. In one aspect, the therapeutic virus expresses an antigen binding peptide of the invention. In another aspect, the antigen binding peptide is attached to the oncolytic

Each of the sequences referred to above may be found in Table C below.

TABLE C

| SEQ ID NO: | Sequence |
|---|---|
| 1 (7A9 HC | INAMN |
| CDR1) | |
| 2 (7A9 HC | RIRSKSNNYATYYADSVKD |
| CDR2) | |
| 3 (7A9 HC | DITRAGYFDV |
| CDR3) | |
| 4 (7A9 LC | SASSSVIYMH |
| CDR1) | |
| 5 (7A9 LC | STSNLAS |
| CDR2) | |
| 6 (7A9 LC | QQRSSYPLT |
| CDR3) | |
| 7 (6F8 HC | TSAMN |
| CDR1) | |
| 8 (6F8 HC | RIRSKNYNYATYYADSVKD |
| CDR2) | |
| 9 (6F8 HC | PPGFAY |
| CDR3) | |
| 10 (6F8 LC | RTSQEISGHLS |
| CDR1) | |
| 11 (6F8 LC | AASSLDS |
| CDR2) | |
| 12 (6F8 LC | LQYSSYPYT |
| CDR3) | |
| 13 (7A9 | EVQLVETGGGLVQPKGSLKLSCAVSGITFNINAMNWVRQAPGK |
| HC) | GLEWVARIRSKSNNYATYYADSVKDRFTISRDDSQSMLYLQMN |
| | NLKTEDTAMYYCVRDITRAGYFDVWGAGTTVTVSS |
| 14 (7A9 LC) | QIVLTQSPAIMSASPGEKVTITCSASSSVIYMHWFQQKPGTSPKL |
| | WIYSTSNLASGVPARFSGSGSGTSYSLTISRMEAEDAATYYCQQ |
| | RSSYPLTFGAGTKLELK |
| 15 (6F8 HC) | EVQLVESGGGLVRPKGSLKLSCAASGFTLNTSAMNWVRQVPG |
| | KGLEWVGRIRSKNYNYATYYADSVKDRFTIFRDDSQNMLYLQM |
| | NNLKTEDTAMYYCVRPPGFAYWGQGTLVTVSA |
| 16 (6F8 LC) | DIQMTQSPSSLSASLGERVSLTCRTSQEISGHLSWLQQKPDGTI |
| | KRLIYAASSLDSGVPKRFSGSRSGSDYSLTISSLESEDFADYYCL |
| | QYSSYPYTFGGGTKLEIK |
| 17 (7A9 HC | GAGGTGCAGCTTGTTGAGACTGGTGGAGGATTGGTGCAGCC |
| NT) | TAAAGGGTCATTGAAACTCTCATGTGCAGTCTCTGGAATCAC |
| | CTTCAATATCAATGCCATGAACTGGGTCCGCCAGGCTCCAGG |
| | AAAGGGTTTGGAATGGGTTGCTCGCATAAGAAGTAAAAGTAA |
| | TAATTATGCAACATATTATGCCGATTCAGTGAAGGACAGGTTC |
| | ACCATCTCCAGAGATGATTCACAAAGCATGCTCTATCTGCAAA |
| | TGAACAACTTGAAAACTGAGGACACAGCCATGTATTACTGTGT |
| | GAGAGATATAACTCGGGCCGGGTACTTCGATGTCTGGGGCG |
| | CAGGGACCACGGTCACCGTCTCCTCA |
| 18 (7A9 LC | CAAATTGTTCTCACCCAGTCTCCAGCAATCATGTCTGCATCTC |
| NT) | CAGGGGAGAAGGTCACCATAACCTGCAGTGCCAGCTCAAGT |
| | GTAATTTACATGCACTGGTTCCAGCAGAAGCCAGGCACTTCT |
| | CCCAAACTCTGGATTTATAGCACATCCAACCTGGCTTCTGGA |
| | GTCCCTGCTCGCTTCAGTGGCAGTGGATCTGGGACCTCTTAC |
| | TCTCTCACAATCAGCCGAATGGAGGCTGAAGATGCTGCCACT |
| | TATTACTGCCAGCAAAGGAGTAGTTATCCGCTCACGTTCGGT |
| | GCTGGGACCAAGCTGGAGCTGAAA |
| 19 (6F8 HC | GAGGTGCAGCTTGTTGAGTCTGGTGGAGGATTGGTGCGGCC |
| NT) | TAAAGGGTCATTGAAACTCTCATGTGCAGCCTCTGGATTCAC |
| | CCTCAATACCTCCGCCATGAACTGGGTCCGCCAGGTTCCAG |
| | GAAAGGGTTTGGAATGGGTTGGTCGCATAAGAAGTAAAAATT |
| | ATAATTATGCAACATATTATGCCGATTCAGTGAAAGACAGGTT |
| | CACCATTTTCAGAGATGATTCACAAAACATGCTCTATCTGCAA |
| | ATGAACAACTTGAAAACTGAGGACACAGCCATGTATTACTGT |
| | GTGAGGCCCCCCGGGTTTGCTTACTGGGGCCAAGGGACTCT |
| | GGTCACTGTCTCTGCA |
| 20 (6F8 LC | GACATCCAGATGACCCAGTCTCCATCCTCCTTATCTGCCTCT |
| NT) | CTGGGAGAAAGAGTCAGTCTCACTTGTCGGACAAGTCAGGAA |
| | ATTAGTGGTCACTTAAGCTGGCTTCAGCAGAAACCAGATGGA |
| | ACTATTAAACGCCTGATCTACGCCGCATCCAGTTTAGATTCTG |
| | GTGTCCCAAAAAGGTTCAGTGGCAGTAGGTCTGGGTCAGATT |
| | ATTCTCTCACCATCAGCAGCCTTGAGTCTGAAGATTTTGCAGA |
| | CTATTACTGTCTACAATATTCTAGTTATCCGTACACGTTCGGA |
| | GGGGGGACCAAGCTGGAAATAAAA |
| 21 | GGTFSSYA |
| (humIGHV182 | |
| CDR1) | |
| 22 | ISAYNGNT |
| (humIGHV182 | |
| CDR2) | |
| 23 | ARDPPLRYFDWFRGSDWFDP |
| (humIGHV182 | |
| CDR3) | |
| 24 | SGSIASNY |
| (humIGLV048 | |
| CDR1) | |
| 25 | EDN |
| (humIGLV048 | |
| CDR2) | |
| 26 | QSYDSTNGV |
| (humIGLV048 | |
| CDR3) | |
| 27 | QMQLVQSGAEVKKPGSSVKVSCKASGGTFSSYAISWVRQAPG |
| (humIGHV182) | QGLEWMGWISAYNGNTNYAQKLQGRVTMTTDTSTSTAYMELR |
| | SLRSDDTAVYYCARDPPLRYFDWFRGSDWFDPWGQGTLVTVS |
| 28 | NFMLTQPHSVSESPGKTVTISCTRSSGSIASNYVQWYQQRPGS |
| (humIGLV048) | SPTTVIYEDNQRPSGVPDRFSGSIDSSSNSASLTISGLKTEDEAD |
| | YYCQSYDSTNGVFGGGTKLTVL |
| 29 | GYTFTDYA |
| (humIGHV062 | |
| CDR1) | |
| 30 | INPGSGNT |
| (humIGHV062 | |
| CDR2) | |
| 31 | ARDRQGPDY |
| (humIGHV062 | |
| CDR3) | |
| 32 | NIGSKN |
| (humIGLV083 | |
| CDR1) | |
| 33 | GDS |
| (humIGLV083 | |
| CDR2) | |
| 34 | QVWDSNSDHPFV |
| (humIGLV083 | |
| CDR3) | |
| 35 | WVQLVESGPEVKKPGASVNVSCKASGYTFTDYAIHWVRQAPG |
| (humIGHV062) | QRPEWMGWINPGSGNTKYSQKFQVRVTITRDTSASTAYMEMT |
| | SLRSEDTAIYYCARDRQGPDYWGQGTLVTVS |
| 36 | SYELMQPPSVSMAPGQTARITCGGNNIGSKNVHWYQQKSGQA |
| (humIGLV083) | PVLVVHGDSDRPSGIPERISGRNFGNTATLTINRVEAGDEADYY |
| | CQVWDSNSDHPFVFGTGTKVTVL |
| 37 | GGTFSNPV |
| (humIGHV237 | |
| CDR1) | |
| 38 | IITMFGTT |
| (humIGHV237 | |
| CDR2) | |
| 39 | AKDPPMFDY |
| (humIGHV237 | |
| CDR3) | |
| 40 | QSISSY |
| (humIGLV120 | |
| CDR1) | |
| 41 | DAS |
| (humIGLV120 | |
| CDR2) | |
| 42 | QQYDNLLALT |
| (humIGLV120 | |
| CDR3) | |
| 43 | QVQLVQSGAEVKKPGSSVKVSCKSSGGTFSNPVISWVRQAPG |
| (humIGHV237) | QGLEWMGGIITMFGTTYYGNSVRGRFIVSRDNSKNMLFLQMNS |
| | LRAEDTAVYYCAKDPPMFDYVVGQGTLVTVS |
| 44 | DIQLTQSPSSLSASVGDRVTITCRASQSISSYLNWYQQKPGKAP |
| (humIGLV120) | KLLIYDASNLETGVPSRFSGSGSGTDFTFTISSLQPEDIATYYCQ |
| | QYDNLLALTFGGGTKVDIK |
| 45 | GGYIDRYF |
| (humIGHV062 | |
| CDR1) | |
| 46 | IYTSGRT |
| (humIGHV062 | |
| CDR2) | |
| 47 | ARDRQGPDY |
| (humIGHV168 | |
| CDR3) | |
| 48 | QGIRND |
| (humIGLV098 | |
| CDR1) | |
| 49 | AAS |
| (humIGLV098 | |
| CDR2) | |
| 50 | LQHNTYPWT |
| (humIGLV098 | |
| CDR3) | |
| 51 | EVQLVETGPGLVKPSQTLSLTCSVSGGYIDRYFIHWVRQAPGQR |
| (humIGHV062) | PEWMGWIYTSGRTKYSQKFQVRVTITRDTSASTAYMEMTSLRS |
| | EDTAIYYCARDRQGPDYWGQGTLVTVS |
| 52 | DIVMTQSPSSLSASVGDRVTITCRASQGIRNDLGWYQQKPGKA |
| (humIGLV098) | PKRLIYAASSLQSGVPSRFSGSGSGTEFTLTISSLQPEDFATYYC |
| | LQHNTYPWTFGQGTKVEIK |
| 53 | CISKLYGSAGPPPTGEEDTAE |
| (antigen | |
| sequence) | |
| 54 | ISKLYGSAGPPPTGEEDTAE |
| (Biopanning | |
| peptide) | |
| 55 | ATGCTGTTGGGGCTGAAGTGGGTTTGCTTTGTTGTTTTTTATC |
| 6F8 HC | AAGGTGTGCATTGTGAGGTGCAGCTTGTTGAGTCTGGTGGAG |
| w/signal | GATTGGTGCGGCCTAAAGGGTCATTGAAACTCTCATGTGCAG |
| sequence | CCTCTGGATTCACCCTCAATACCTCCGCCATGAACTGGGTCC |
| | GCCAGGTTCCAGGAAAGGGTTTGGAATGGGTTGGTCGCATA |
| | AGAAGTAAAAATTATAATTATGCAACATATTATGCCGATTCAGT |
| | GAAAGACAGGTTCACCATTTTCAGAGATGATTCACAAAACATG |
| | CTCTATCTGCAAATGAACAACTTGAAAACTGAGGACACAGCC |
| | ATGTATTACTGTGTGAGGCCCCCCGGGTTTGCTTACTGGGGC |
| | CAAGGGACTCTGGTCACTGTCTCTGCA |
| 56 | ATGAGGGTTCCTGCTCACGTTTTTGGCTTCTTGTTGCTCTGGT |
| 6F8 LC | TTCCAGGTACCAGATGTGACATCCAGATGACCCAGTCTCCAT |
| w/signal | CCTCCTTATCTGCCTCTCTGGGAGAAAGAGTCAGTCTCACTT |
| sequence | GTCGGACAAGTCAGGAAATTAGTGGTCACTTAAGCTGGCTTC |
| | AGCAGAAACCAGATGGAACTATTAAACGCCTGATCTACGCCG |
| | CATCCAGTTTAGATTCTGGTGTCCCAAAAAGGTTCAGTGGCA |
| | GTAGGTCTGGGTCAGATTATTCTCTCACCATCAGCAGCCTTG |
| | AGTCTGAAGATTTTGCAGACTATTACTGTCTACAATATTCTAG |
| | TTATCCGTACACGTTCGGAGGGGGGACCAAGCTGGAAATAAA |
| | A |
| 57 | MLLGLKWVCFVVFYQGVHCEVQLVESGGGLVRPKGSLKLSCAA |
| 6F8 HC | SGFTLNTSAMNWVRQVPGKGLEWVGRIRSKNYNYATYYADSV |
| w/signal | KDRFTIFRDDSQNMLYLQMNNLKTEDTAMYYCVRPPGFAYWG |
| sequence | QGTLVTVSA |
| 58 | MRVPAHVFGFLLLWFPGTRCDIQMTQSPSSLSASLGERVSLTC |
| 6F8 LC | RTSQEISGHLSWLQQKPDGTIKRLIYAASSLDSGVPKRFSGSRS |
| w/signal | GSDYSLTISSLESEDFADYYCLQYSSYPYTFGGGTKLEIK |
| sequence | |
| 59 | ATGCTGTTGGGGCTGAAGTGGGTTTTCTTTGTTGTTTTTTATC |
| 7A9 HC | AAGGTGTGCATTGTGAGGTGCAGCTTGTTGAGACTGGTGGA |
| w/signal | GGATTGGTGCAGCCTAAAGGGTCATTGAAACTCTCATGTGCA |
| sequence | GTCTCTGGAATCACCTTCAATATCAATGCCATGAACTGGGTC |
| | CGCCAGGCTCCAGGAAAGGGTTTGGAATGGGTTGCTCGCAT |
| | AAGAAGTAAAAGTAATAATTATGCAACATATTATGCCGATTCA |
| | GTGAAGGACAGGTTCACCATCTCCAGAGATGATTCACAAAGC |
| | ATGCTCTATCTGCAAATGAACAACTTGAAAACTGAGGACACA |
| | GCCATGTATTACTGTGTGAGAGATATAACTCGGGCCGGGTAC |
| | TTCGATGTCTGGGGCGCAGGGACCACGGTCACCGTCTCCTC |
| | A |
| 60 | ATGCATTTTCAAGTGCAGATTTTCAGCTTCCTGCTAATCAGTG |
| 7A9 HC | CCTCAGTCATAATGTCCAGAGGACAAATTGTTCTCACCCAGT |
| w/signal | CTCCAGCAATCATGTCTGCATCTCCAGGGGAGAAGGTCACCA |
| sequence | TAACCTGCAGTGCCAGCTCAAGTGTAATTTACATGCACTGGTT |
| | CCAGCAGAAGCCAGGCACTTCTCCCAAACTCTGGATTTATAG |
| | CACATCCAACCTGGCTTCTGGAGTCCCTGCTCGCTTCAGTGG |
| | CAGTGGATCTGGGACCTCTTACTCTCTCACAATCAGCCGAAT |
| | GGAGGCTGAAGATGCTGCCACTTATTACTGCCAGCAAAGGAG |
| | TAGTTATCCGCTCACGTTCGGTGCTGGGACCAAGCTGGAGCT |
| | GAAA |
| 61 | MLLGLKWVFFVVFYQGVHCEVQLVETGGGLVQPKGSLKLSCAV |
| 7A9 HC | SGITFNINAMNWVRQAPGKGLEWVARIRSKSNNYATYYADSVK |
| w/signal | DRFTISRDDSQSMLYLQMNNLKTEDTAMYYCVRDITRAGYFDV |
| sequence | WGAGTTVTVSS |
| 62 | MHFQVQIFSFLLISASVIMSRGQIVLTQSPAIMSASPGEKVTITCS |
| 7A9 HC | ASSSVIYMHWFQQKPGTSPKLWIYSTSNLASGVPARFSGSGSG |
| w/signal | TSYSLTISRMEAEDAATYYCQQRSSYPLTFGAGTKLELK |
| sequence | |
| 63 | EEKIEWLESHQD |
| Epitope | |
| mapping | |
| sequence 1 | |
| 64 | ESHQDADIEDFK |
| Epitope | |
| mapping | |
| sequence 2 | |
| 65 | IEDFKAKKKELE |
| Epitope | |
| mapping | |
| sequence 3 | |
| 66 | KKELEEIVQPII |
| Epitope | |
| mapping | |
| sequence 4 | |
| 67 | VQPIISKLYGSA |
| Epitope | |
| mapping | |
| sequence 5 | |
| 68 | LYGSAGPPPTGE |
| Epitope | |
| mapping | |
| sequence 6 | |
| 69 | PPTGEEDTAEKD |
| Epitope | |
| mapping | |
| sequence 7 | |
| 70 | ISKLYGSAEDTAE |
| 7A9 epitope | |
| 71 | PPTGEEDTAE |
| 6F8 epitope | |
| 72 | X1-X2-A-M-N, wherein X1 is a hydrophobic or polar neutral side |
| HC CDR1 | chain amino acid and X2 is a polar neutral side chain amino acid |
| 73 | R-I-R-S-K-X1-X2-N-Y-A-T-Y-Y-A-D-S-N-K-D, wherein X1 is a |
| HC CDR2 | polar neutral side chain amino acid; and X2 is a hydrophobic or |
| | polar neutral side chain amino acid |
| 74 | x-x-X1-S-X2-x-I-X3-x-H-x-x, wherein x is any amino acid; X1 is a |
| LC CDR1 | polar neutral side chain amino acid; X2 is a polar neutral side |
| | chain amino acid; and X3 is a hydrophobic or polar neutral side |
| | chain amino acid |
| 75 | X1-X2-S-X3-L-x-S, wherein x is any amino acid; X1 is a |
| LC CDR2 | hydrophobic or polar neutral side chain amino acid; X2 is a |
| | hydrophobic or polar neutral side chain amino acid; and X3 is a |
| | polar neutral side chain amino acid |
| 76 | X1-Q-X2-S-S-Y-P-X3-T, wherein x is any amino acid; X1 is a |
| LC CDR3 | hydrophobic or polar neutral side chain amino acid; and X3 is a |
| | polar neutral side chain amino acid |

### II. Method of Use

In another aspect, an isolated antibody of the present invention, as described above, may be used in treating and preventing cancer and associated diseases in a subject. The antibodies of the present invention may be conjugated to radioisotopes or chemotherapeutic compounds in order to provide specific delivery of radiation and chemotherapy to the site of a tumor. Further, the antibodies of the present invention may be part of a combination therapy. Preferably, a combination therapy would include the use of the antibody of the present invention along with a radiation therapy or chemotherapy course of treatment. It has also been suggested that monoclonal antibodies, such as those described herein, may increase the susceptibility of tumor cells to the effects of chemotherapy or radiation. In preferred embodiments, the antibodies of the invention may be used to enhance the efficacy of cancer radiotherapy.

In one aspect, the present disclosure provides a method of determining a dose of radiation exposure of a subject or biological sample, for example biological dosimetry. Biological dosimetry refers to the use of tests of biological matter to determine the dose it was exposed to. It is especially useful in radiation accidents when physical dosimetry (eg. TLD or ionization chamber) is not possible. In some embodiments, the antigen binding peptides of the disclosure are used to determine the dose of radiation exposure in a subject.

In yet another aspect, the present disclosure provides a method of imaging a cancer. As such, an antibody of the invention may be conjugated to an imaging agent. For instance, an scFv may be conjugated to an imaging agent. Suitable imaging agents may include, but are not limited to, imaging/tracking agents that may be used for microscopy, e.g. fluorescent microscopy, confocal microscopy, or electron microscopy, magnetic resonance imaging, tomography, such as gamma (SPECT/CT, planar) and positron emission tomography (PET/CT), radiography, or ultrasound. Imaging/tracking agents may be detectable in situ, in vivo, ex vivo, and in vitro. In general, imaging/tracking agents may include luminescent molecules, chemiluminescent molecules, fluorochromes, fluorescent quenching agents, colored molecules, radioisotopes, scintillants, massive labels (for detection via mass changes), biotin, avidin, streptavidin, protein A, protein G, antibodies or fragments thereof, Grb2, polyhistidine, Ni²⁺, Flag tags, myc tags, heavy metals, zirconium (binds to desferoxamine), enzymes, alkaline phosphatase, peroxidase, luciferase, electron donors/acceptors, acridinium esters, and colorimetric substrates. The skilled artisan would readily recognize other useful labels that are not mentioned above, which may be employed in the operation of the present invention.

The antibodies are as described in Section I above. The subject, the cancer, and the administration of the antibodies are described below.

(a) Subject

A method of the invention may be used to detect or treat a tumor in a subject that is a human, a livestock animal, a companion animal, a lab animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In preferred embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. The genotype of the sterile animal can and may vary depending on the intended use of the animal. In embodiments where the animal is a mouse, the mouse may be a C57BL/6 mouse, a Balb/c mouse, a 129sv mouse, a GL261 tumor bearing mouse, a D54 tumor bearing mouse, or any other laboratory strain.

(b) Tumor

An antibody of the invention may be used to treat or recognize tumor derived from a neoplasm or a cancer. The neoplasm may be malignant or benign, the cancer may be primary or metastatic; the neoplasm or cancer may be early stage or late stage. Non-limiting examples of neoplasms or cancers that may be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Meningioma, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood). In preferred embodiments, the neoplasm or cancer is non-small cell lung carcinoma.

(c) Administration

In certain aspects, a pharmacologically effective amount of an antibody of the invention, including immunologically reactive fragments, may be administered to a subject. Administration is performed using standard effective techniques, including peripherally or locally. Peripheral administration includes but is not limited to intravenous, intraperitoneal, subcutaneous, pulmonary, transdermal, intramuscular, intranasal, buccal, sublingual, or suppository administration. Local administration, includes administration directly into an anatomical site of interest, for example, administration directly into the central nervous system (CNS) which includes but is not limited to via a lumbar, intraventricular or intraparenchymal catheter or using a surgically implanted controlled release formulation. In some embodiments, the antigen binding peptides as disclosed herein are used in combination with focused ultrasound. Focused ultrasound is an early-stage, non-invasive therapeutic technology with the potential to transform the treatment of many medical disorders by using ultrasonic energy to target tissue deep in the body without incisions or radiation. High-intensity focused ultrasound (HIFU) is a non-invasive therapeutic technique that uses non-ionizing ultrasonic waves to heat tissue. HIFU can be used to increase the flow of blood or lymph, or to destroy tissue, such as tumors, through a number of mechanisms. HIFU may be combined with other imaging techniques such as medical ultrasound or MRI to enable guidance of the treatment and monitoring.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. It may be particularly useful to alter the solubility characteristics of the antibodies useful in this discovery, making them more lipophilic, for example, by encapsulating them in liposomes or by blocking polar groups.

Effective peripheral systemic delivery by intravenous or intraperitoneal or subcutaneous injection is a preferred method of administration to a living patient. Suitable vehicles for such injections are straightforward. In addition, however, administration may also be effected through the mucosal membranes by means of nasal aerosols or suppositories. Suitable formulations for such modes of administration are well known and typically include surfactants that facilitate cross-membrane transfer. Such surfactants are often derived from steroids or are cationic lipids, such as N-[1-(2,3-dioleoyl)propyl]-N,N,N-trimethyl ammonium chloride (DOTMA) or various compounds such as cholesterol hemisuccinate, phosphatidyl glycerols and the like. Compositions of the disclosure may also include chelators such as, in non-limiting examples, tetracarboxylic acid (DOTA), 1,4,7-triazacyclononane-N,N′,N″-triacetic acid (NOTA) and desferoxamine.

The concentration of antibody in formulations to be administered is an effective amount and ranges from as low as about 0.1% by weight to as much as about 15 or about 20% by weight and will be selected primarily based on fluid volumes, viscosities, and so forth, in accordance with the particular mode of administration selected if desired. A typical composition for injection to a living patient could be made up to contain 1 mL sterile buffered water of phosphate buffered saline and about 1-1000 mg of any one of or a combination of the humanized antibody of the present discovery. The formulation could be sterile filtered after making the formulation, or otherwise made microbiologically acceptable. A typical composition for intravenous infusion could have volumes between 1-250 mL of fluid, such as sterile Ringer's solution, and 1-100 mg per ml, or more in anti-GRP78 antibody concentration. Therapeutic agents of the discovery can be frozen or lyophilized for storage and reconstituted in a suitable sterile carrier prior to use. Lyophilization and reconstitution may lead to varying degrees of antibody activity loss (e.g. with conventional immune globulins, IgM antibodies tend to have greater activity loss than IgG antibodies). Dosages administered are effective dosages and may have to be adjusted to compensate. The pH of the formulations generally pharmaceutical grade quality, will be selected to balance antibody stability (chemical and physical) and comfort to the patient when administered. Generally, a pH between 4 and 8 is tolerated. Doses will vary from individual to individual based on size, weight, and other physiobiological characteristics of the individual receiving the successful administration.

As used herein, the term “effective amount” means an amount of a substance such as a compound that leads to measurable and beneficial effects for the subject administered the substance, i.e., significant efficacy. The effective amount or dose of compound administered according to this discovery will be determined by the circumstances surrounding the case, including the compound administered, the route of administration, the status of the symptoms being treated and similar patient and administration situation considerations among other considerations.

In some embodiments, when the antibody is an anti-GRP78 labeled with ⁶⁴Cu or Zirconium, the dose administered may be about 0.01, 0.02, 0.03, 0.04, 0.05 0.06, 0.07, 0.08, 0.09, 0.1, 0.011, 0.012, 0.013, 0.013, 0.014, 0.015, 0.016, 0.017, 0.018, 0.019, 0.02, 0.021, 0.022, 0.023, 0.023, 0.024, 0.025, 0.026, 0.027, 0.028, 0.029, 0.03, 0.031, 0.032, 0.033, 0.033, 0.034, 0.035, 0.036, 0.037, 0.038, 0.039, 0.04, 0.041, 0.042, 0.043, 0.043, 0.044, 0.045, 0.046, 0.047, 0.048, 0.049, 0.05, 0.051, 0.052, 0.053, 0.053, 0.054, 0.055, 0.056, 0.057, 0.058, 0.059, 0.06, 0.061, 0.062, 0.063, 0.063, 0.064, 0.065, 0.066, 0.067, 0.068, 0.069, 0.07, 0.071, 0.072, 0.073, 0.073, 0.074, 0.075, 0.076, 0.077, 0.078, 0.079, 0.08, 0.081, 0.082, 0.083, 0.083, 0.084, 0.085, 0.086, 0.087, 0.088, 0.089, 0.09, 0.091, 0.092, 0.093, 0.093, 0.094, 0.095, 0.096, 0.097, 0.098, 0.099, or about 0.1 rem/mCi.

The frequency of dosing may be daily or once, twice, three times or more per week or per month, as needed as to effectively treat the symptoms. The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outpatient clinic. Duration of treatment could range from a single dose administered on a one-time basis to a life-long course of therapeutic treatments.

Although the foregoing methods appear the most convenient and most appropriate and effective for administration of proteins such as antibodies, by suitable adaptation, other effective techniques for administration, such as intraventricular administration, transdermal administration and oral administration may be employed provided proper formulation is utilized herein.

In addition, it may be desirable to employ controlled release formulations using biodegradable films and matrices, or osmotic mini-pumps, or delivery systems based on dextran beads, alginate, or collagen.

Typical dosage levels can be determined and optimized using standard clinical techniques and will be dependent on the mode of administration.

### Definitions

As used herein, “antibody” refers to an immunoglobulin derived molecule that specifically recognizes either GRP78. An antibody of the invention may be a full length antibody (IgM, IgG, IgA, IgE) or may be an antibody fragment (Fab, F(ab′)2, scFv). An antibody may be chimeric or may be humanized.

As used herein, “CDR” means “complementary determining region.” CDRs may also be referred to as hypervariable regions.

As used herein, “light chain” is the small polypeptide subunit of the antibody. A typical antibody comprises two light chains and two heavy chains.

As used herein, the “heavy chain” is the large polypeptide subunit of the antibody. The heavy chain of an antibody contains a series of immunoglobulin domains, with at least one variable domain and at least one constant domain.

“Humanized”, as used herein, refers to the process where monoclonal antibodies are produced using recombinant DNA to create constructs capable of expression in human cell culture. Any known techniques for producing these constructs will work for purposes of the present invention.

As used herein, “single chain variable fragments” or “scFv” or “scFvs”, refer to fusion proteins of the variable regions of the heavy and light chains of immunoglobulins connected via a linker. In some embodiment, the linker is a peptide of about 10 to 25 amino acids.

A “therapeutic agent” for purposes of the present invention, refers to an agent that reduces tumor growth, any related cancer growth, or reduces the symptoms associated with cancerous cell growth. The therapeutic agent that is preferably conjugated to the antibody of the present invention is preferably a biologic, pharmaceutical or chemical agent. A non-limiting list of therapeutic agents that may be suitable for use in the present invention is described above.

As used herein, “imaging agent” refers to any agent that can be used to locate and produce an image of cancerous cell growth or tumors. A non-limiting list of imagining agents that may be suitable for use in the present invention is described above.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Monoclonal antibodies against GRP78 were created, and named 6F8 and 7A9. The antigen sequence is SEQ ID NO: 53 (CISKLYGSAGPPPTGEEDTAE (c-terminal peptide)). Immunogens were created using peptide-KLH conjugates and the host strain BALB/c mouse. The 6F8 antibody comprised a heavy chain variable region amino acid sequence of SEQ ID NO:15 (minus the leader sequence) and a light chain variable region amino acid sequence of SEQ ID NO:16 (minus the leader sequence). The 7A9 antibody comprised a heavy chain variable region amino acid sequence of SEQ ID NO:13 (minus the leader sequence) and a light chain variable region amino acid sequence of SEQ ID NO:14 (minus the leader sequence).

Mouse hybridoma generation (4A6O9 and 4A4A4) was performed using the GRP78 protein as the antigen/immunogen using the host strain BALB/c mouse. Lastly, human scFvs (1154, 1164, 1171, 1183) were generated using the Biopanning peptide sequence; SEQ ID NO:54 (ISKLYGSAGPPPTGEEDTAE (c-terminal peptide)).

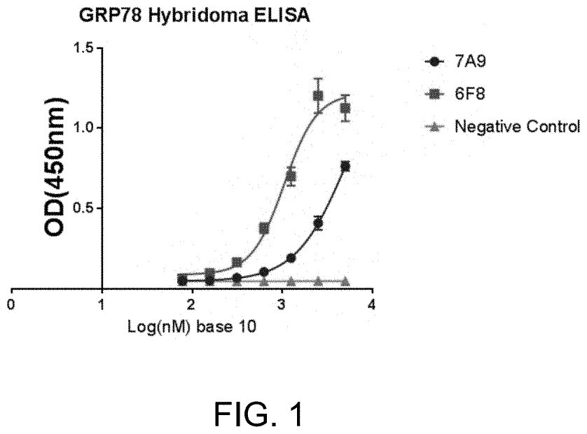

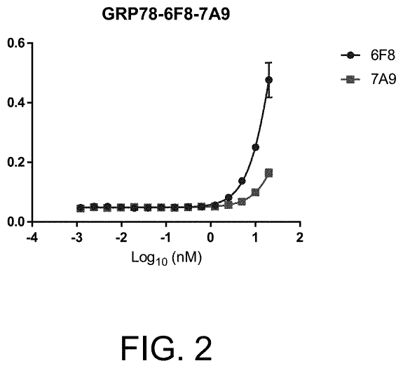

FIG. 1 depicts a graph showing the binding of anti-GRP78 mouse monoclonal antibodies to C-terminal GRP78 peptide in an ELISA assay. FIG. 2 depicts the binding of anti-GRP78 mouse monoclonal antibodies to GRP78 protein in an ELISA assay.

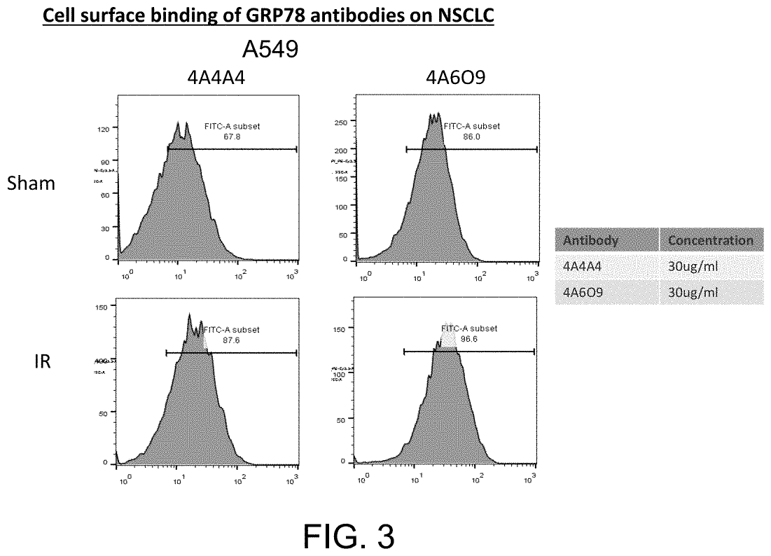

Cell surface binding of GRP78 antibodies: Methods: Irradiation: 3Gy-3 times (6 h and 12 hours apart); 150,000 cells per staining group; Stain with various primary antibodies (1 h); Stain with secondary antibody: Alexafluor 488 anti-mouse IgG (1 h); Data acquisition with flow cytometer; Added PI to each sample immediately before acquisition; and Gated on live cells and acquired 10,000 events. FIG. 3 shows the cell surface binding of GRP78 antibodies on NSCLC. Antibody 4A4A4 and 4A6O9 were used at a concentration of 30 ug/ml.

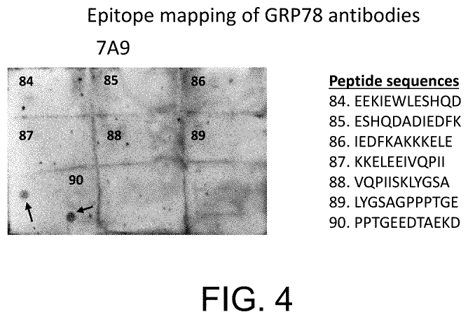

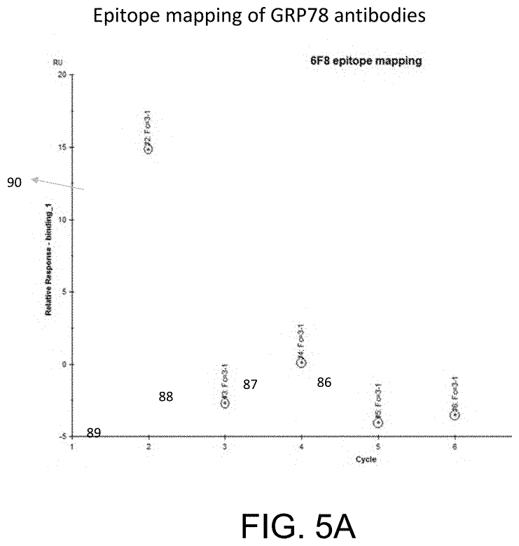

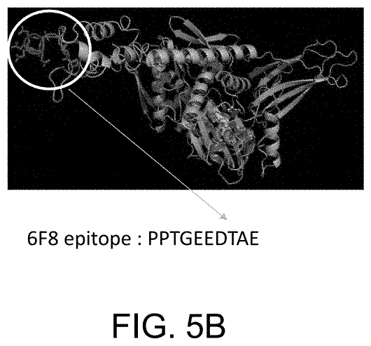

Seven peptides were synthesized from the c-terminus of GRP78. Each peptide was a 12-mer with 5 amino acid overlap. The sequences of the peptides are shown above. Each of the peptide was coated in duplicate on nitrocellulose membrane and incubated with 7A9 antibody. After washing, the blot was developed using chemiluminescence. The arrows indicate positive spot, FIG. 4. 7A9 epitope: SEQ ID NO: 70 (ISKLYGSA . . . EDTAE). FIG. 5A-5B Epitope mapping of 6F8 antibody was performed using Biacore T200. 6F8 was immobilized on the CM5 sensor chip and peptides 86-90 (overlapping peptides in the immunogen region of 6F8) were passed as analyte. Relative binding response (response on blank channel subtracted from the 6F8 immobilized channel) of each peptide to 6F8 was analyzed using the BIAevaluation software. Cut-off for nonbinding peptides is 0 RU. Three-dimensional model of GRP78 protein indicating the epitope sequence on the protein in magenta color. The figure was generated using Pymol software.

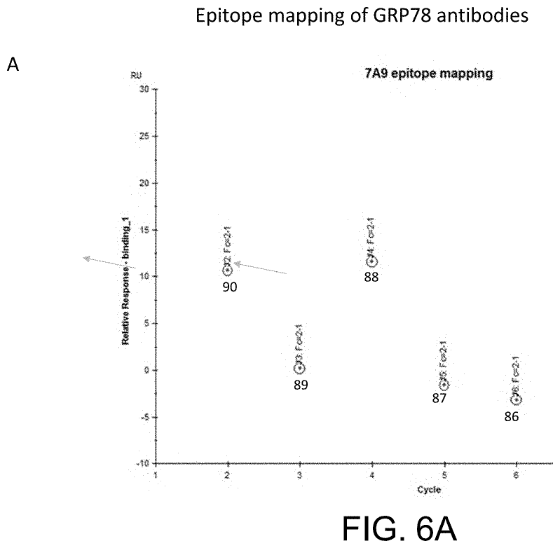

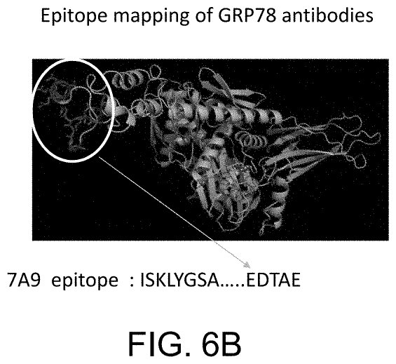

FIG. 6A-6B Epitope mapping of 7A9 antibody was performed using Biacore T200. 7A9 was immobilized on the CM5 sensor chip and peptides 86-90 (overlapping peptides in the immunogen region of 7A9) were passed as analyte. Relative binding response (response on blank channel subtracted from the 7A9 immobilized channel) of each peptide to 7A9 was analyzed using the BIAevaluation software. Cut-off for nonbinding peptides is 0 RU. Three-dimensional model of GRP78 protein indicating the epitope sequence on the protein in red color. The figure was generated using Pymol software.

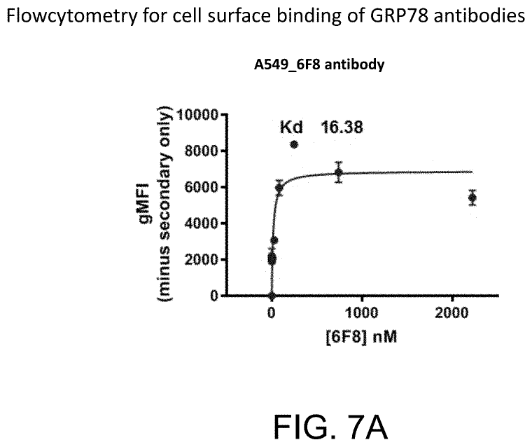

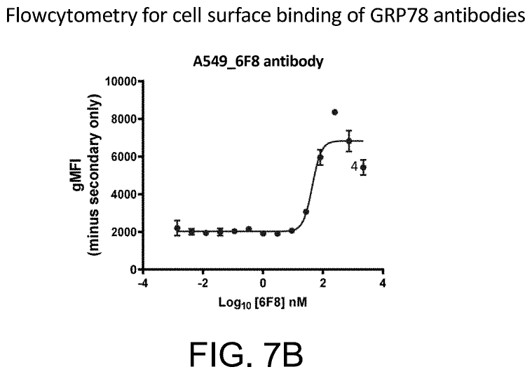

A Flow cytometry assay for cell surface binding of 6F8 antibody to A549 cells was done (FIG. 7A-7B). Binding conditions: 0.1×10⁶ cells incubated with 3-fold dilutions of antibody started at 2.22 μM. Cells were washed 2× with FACS buffer. Stained with secondary antibody. Washed twice and analyzed in FACS buffer by flow cytometer FACS Canto II (BD). Geometric mean fluorescence intensity was fitted using the “One site-specific binding” in Graphpad Prism software. Graph showing the same data as in A fitted using the “Sigmoidal, 4PL, X is log(concentration)” in Graphpad Prism software (FIG. 7B).

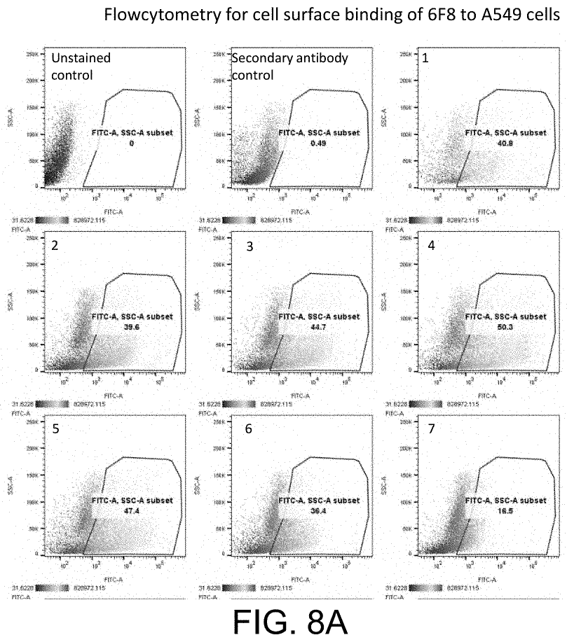

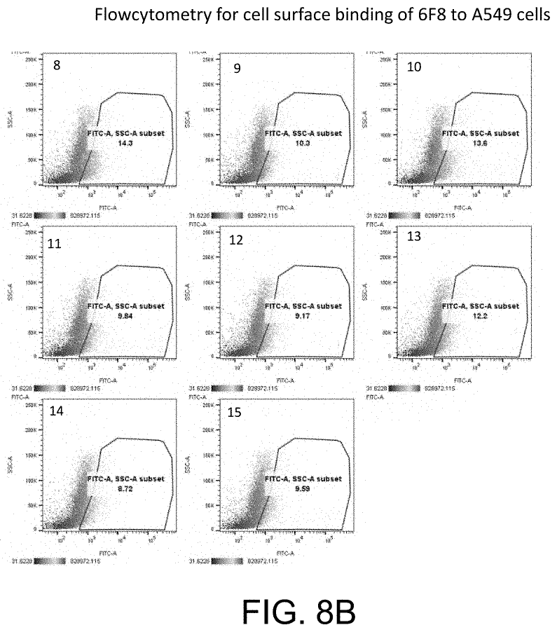

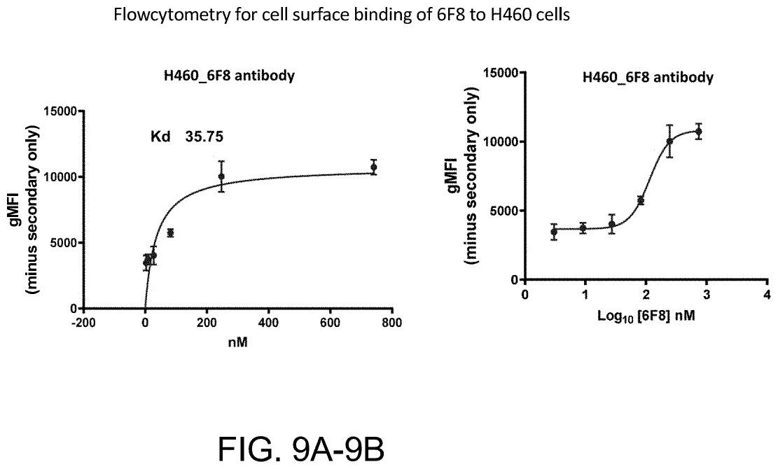

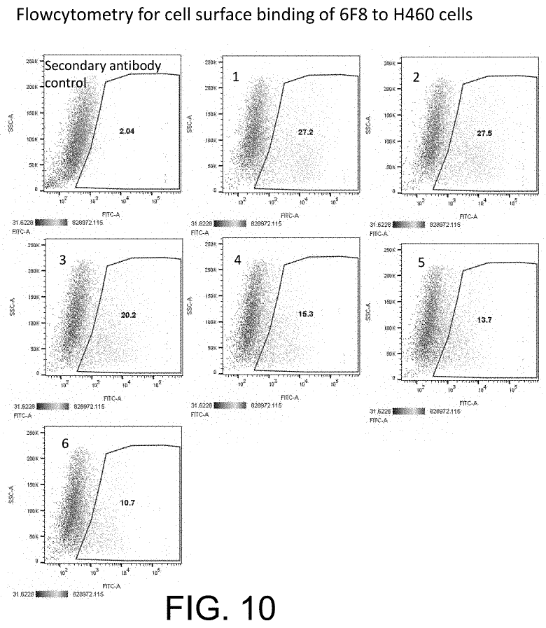

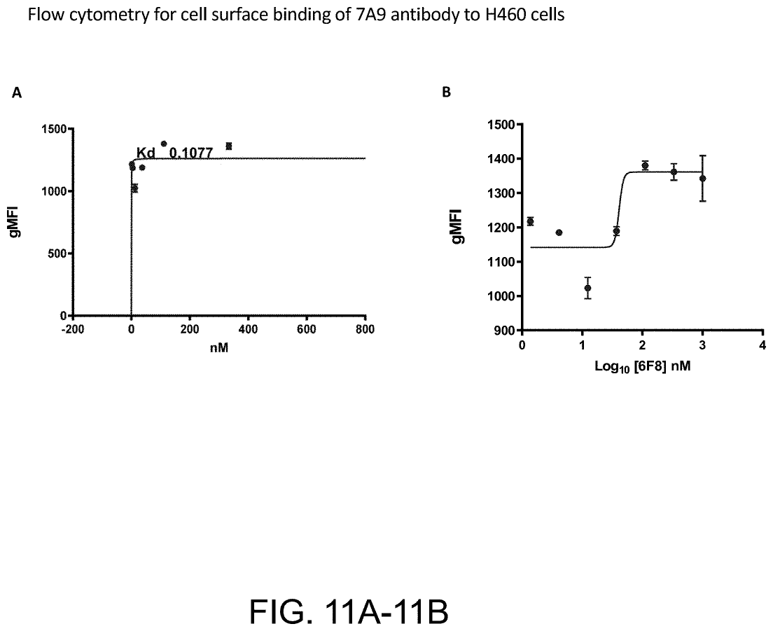

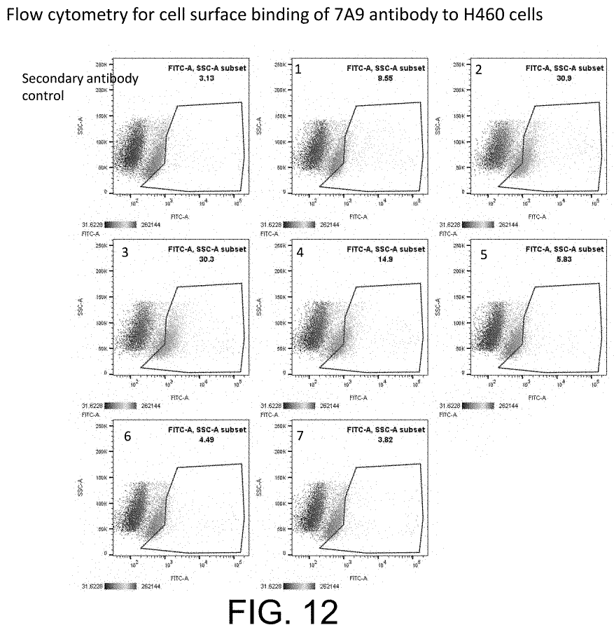

Flowcytometry for cell surface binding of 6F8 to A549 cells was performed and the results are shown in FIG. 8A-8B. Flowcytometry for cell surface binding of 6F8 to H460 cells is shown in FIGS. 9A-9B and FIG. 10. Flow cytometry for cell surface binding of 7A9 antibody to H460 cells is shown in FIGS. 11A-11B and FIG. 12.

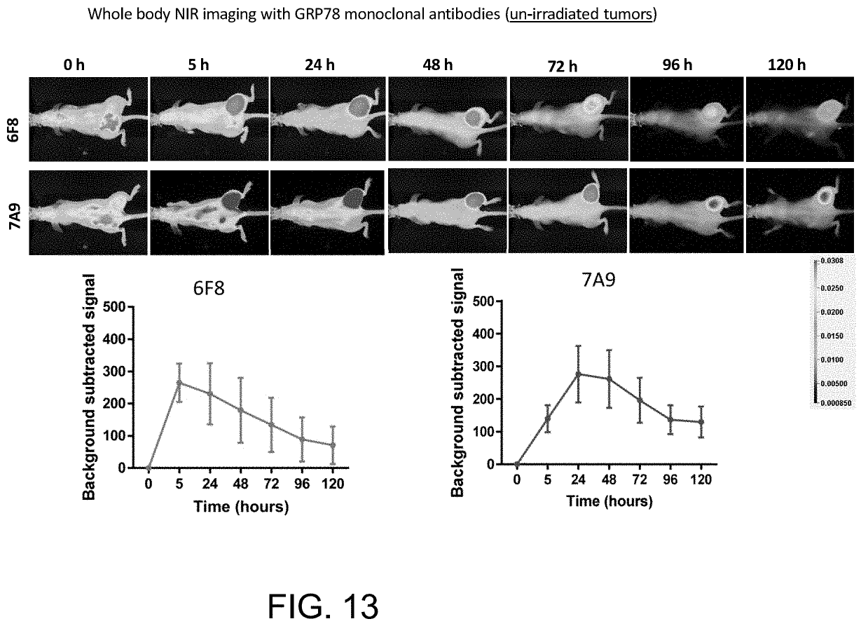

A549 tumors were injected in hind limbs of nude mice. 6F8 and 7A9 antibodies were labeled with the IR dye 800 (Licor). 40 ug of each antibody was injected in the tail vein and imaged every day using the Pearl imager. Whole body NIR imaging with GRP78 monoclonal antibodies (un-irradiated tumors) are shown in FIG. 13.

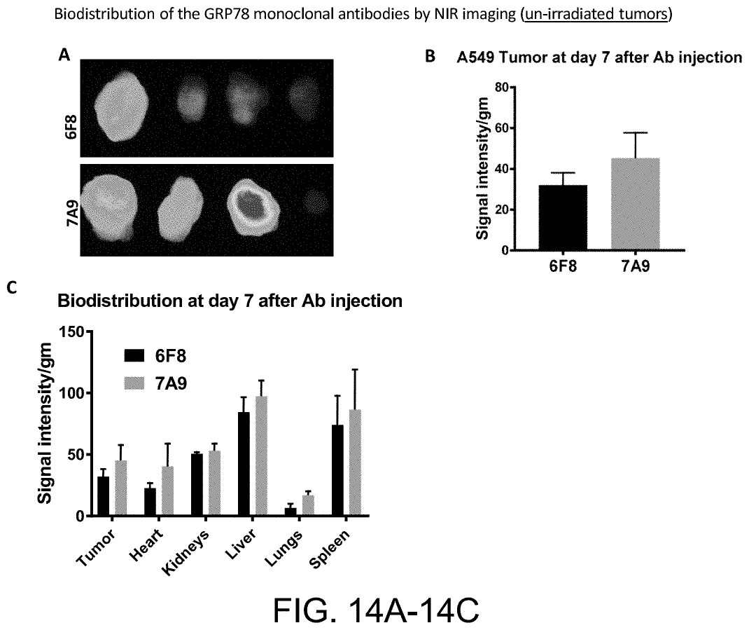

A549 tumors were injected in hind limbs of nude mice. 6F8 and 7A9 antibodies were labeled with the IR dye 800 (Licor). 40 ug of each antibody was injected in the tail vein and imaged every day using the Pearl imager. At day 7 after antibody injection, mice were euthanized and all organs harvested and imaged with the Pearl imager. FIG. 14A shows the images of the harvested A549 tumors. FIG. 14B Bar graph showing signal intensity per gram of the harvested tumors. FIG. 14C signal intensity of all organs per gm of their respective weights.

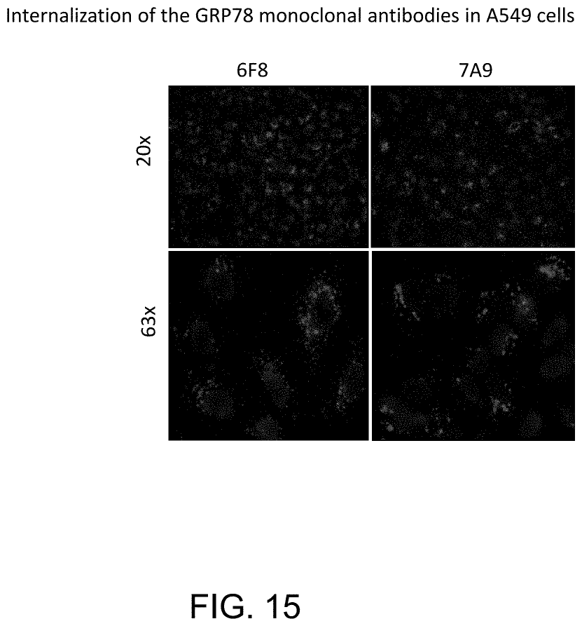

A549 cells were seeded into chamber slides. GRP78 antibodies were labeled with the pHRodo iFL red dye (Thermo Scientific) according to the manufacturer's protocol and added to the cells. After 24 h incubation at 37° C., slides were washed with PBS and stained with nucblue live stain to stain the nucleus. Images were captured in an inverted fluorescent microscope (Zeiss). Presence of red punctate staining indicates endocytosis of the antibodies in the intracellular acidic compartments. Internalization of the GRP78 monoclonal antibodies in A549 cells shown in FIG. 15.

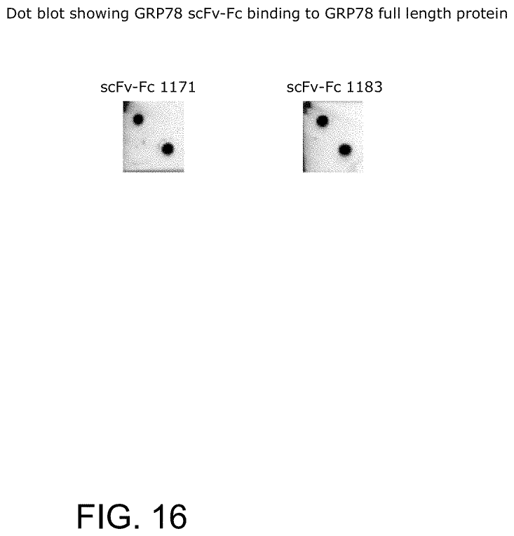

For the dot blot (FIG. 16), recombinant GRP78 full length protein was spotted onto nitrocellulose membrane in duplicate. After blocking, the blot was incubated with anti-GRP78 scFv-Fcs 1171 and 1183. The scFv-Fcs were detected for binding to the recombinant GRP78 protein using anti-human Fc-HRP conjugated antibody.

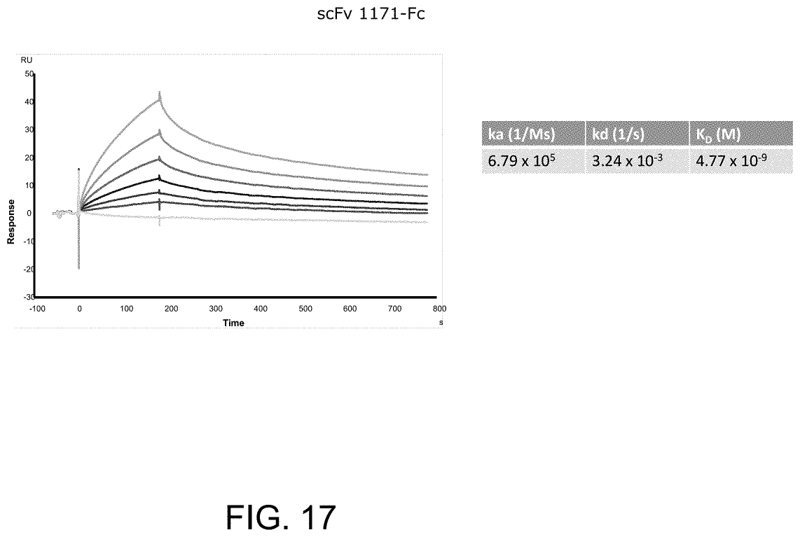

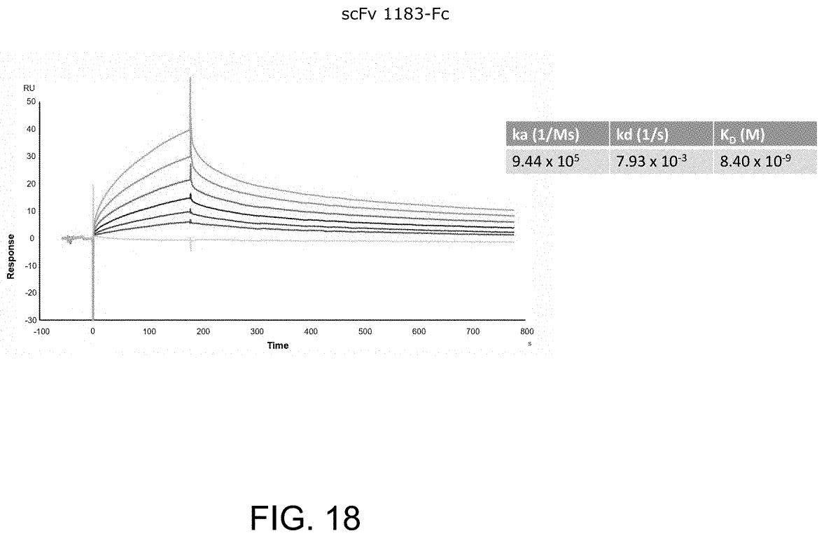

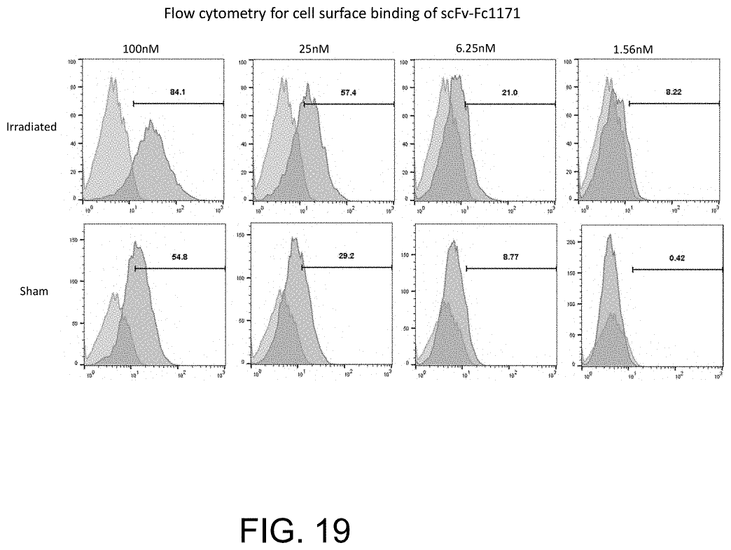

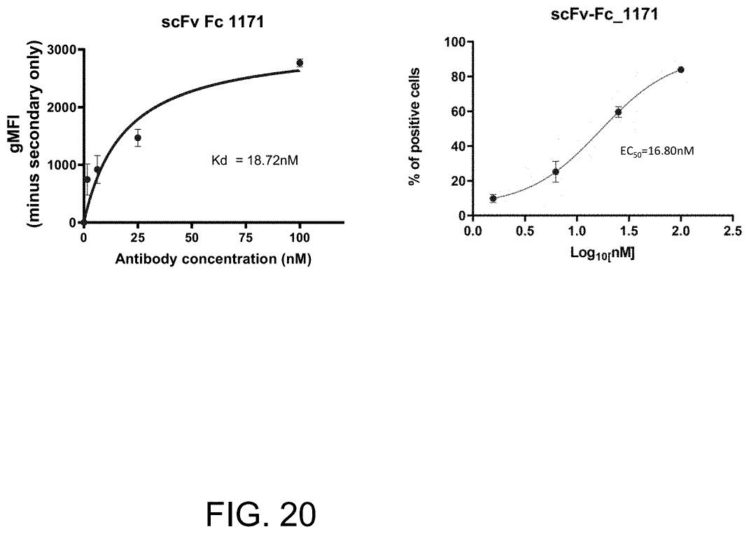

Biacore analysis for binding affinity of anti-GRP78 scFV 1171-Fc to recombinant full length GRP78 protein. The highest concentration of scFv1171-Fc was 500 nM followed by two-fold serial dilutions. The on-, off-rates and dissociation constant are shown in the table (FIG. 17). Biacore analysis for binding affinity of anti-GRP78 scFV 1171-Fc to recombinant full length GRP78 protein. The highest concentration of scFv1183-Fc was 500 nM followed by two-fold serial dilutions. The on-, off-rates and dissociation constant are shown in the table (FIG. 18). A549 cells were either sham or irradiated with 3 doses of 3Gy. Cells were harvested and incubated with indicated concentrations of the scFV-Fc1171 antibodies. Representative overlay histograms (blue: secondary antibody control; red: scFv-Fc1171) are shown (FIG. 19). Flow cytometry assay for cell surface binding of scFv-Fc1171 antibody to irradiated A549 cells. Binding conditions: 0.1×10⁶ cells incubated with 4-fold dilutions of antibody started at 100 nM. Cells were washed 2× with FACS buffer. Stained with secondary antibody. Washed twice and analyzed in FACS buffer by flow cytometer FACS Canto II (BD). Geometric mean fluorescence intensity was fitted using the One site-specific binding in GraphpadPrism software (FIG. 20).

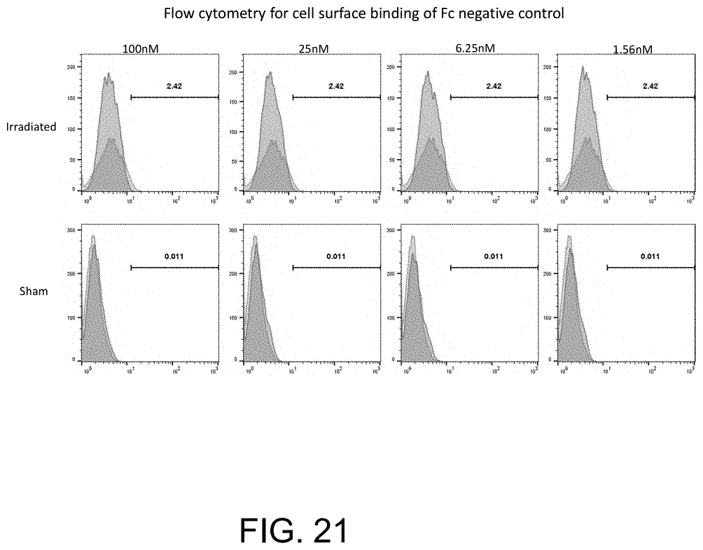

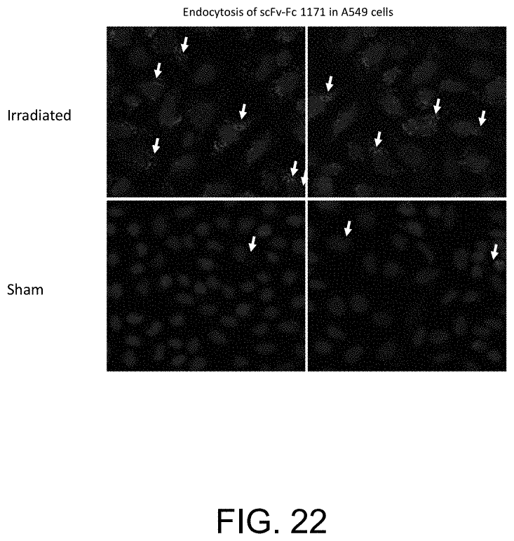

A549 cells were either sham or irradiated with 3 doses of 3Gy. Cells were harvested and incubated with indicated concentrations of the Fc negative control. Representative overlay histograms (blue: secondary antibody control; red: Fc negative control) are shown (FIG. 21). A549 cells were either sham or irradiated with 3 doses of 3Gy. scFv-Fc 1171 was labeled with pHRodo red pH-sensitive dye that fluoresces red in acidic cellular compartments. White arrows indicate the internalized antibody (FIG. 22).

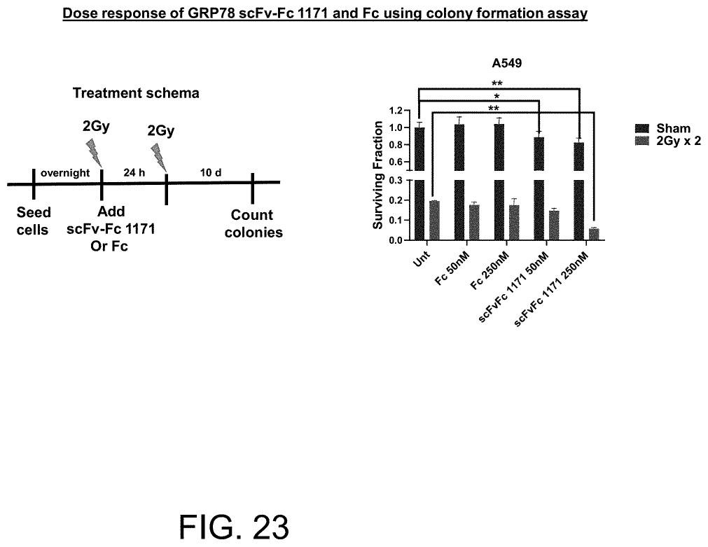

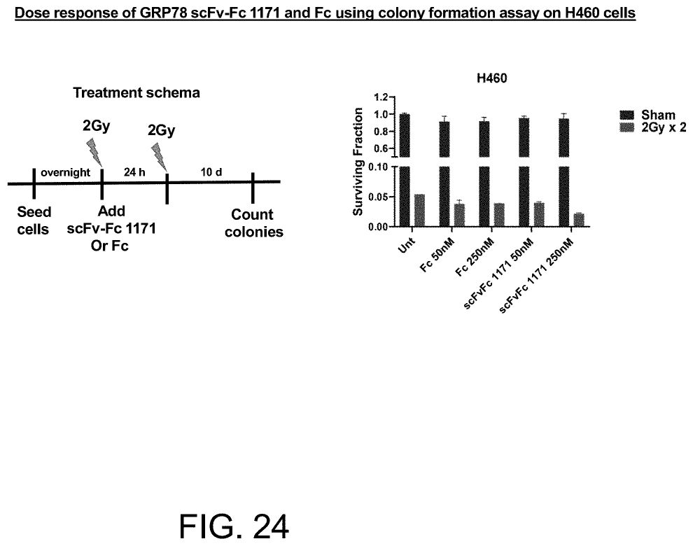

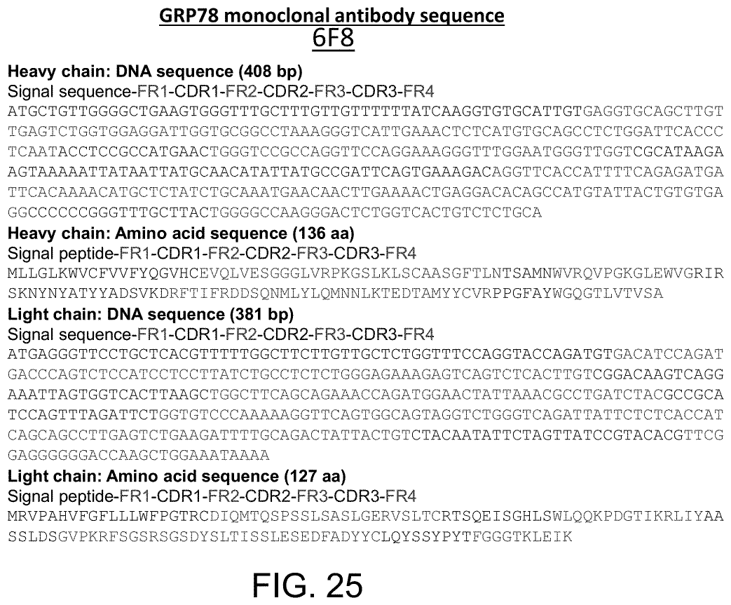

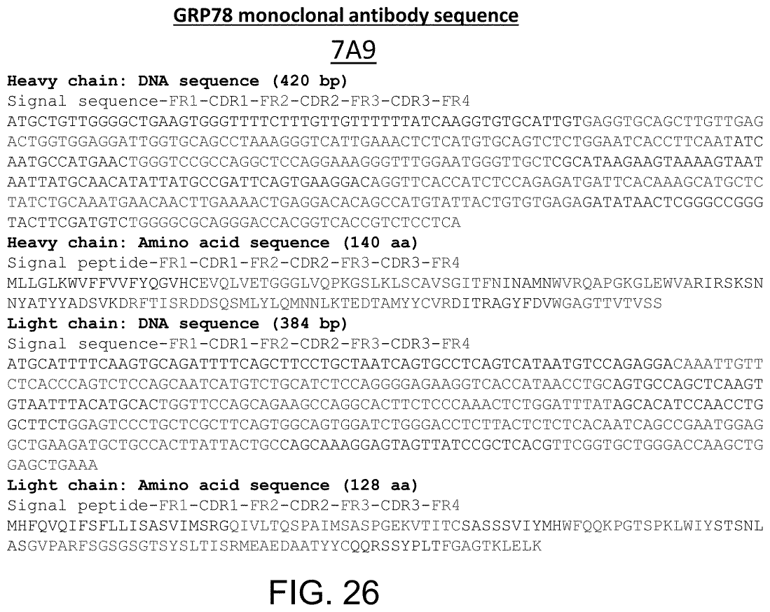

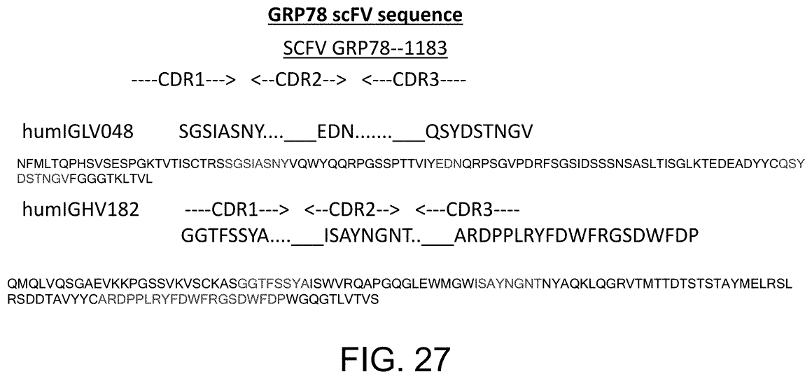

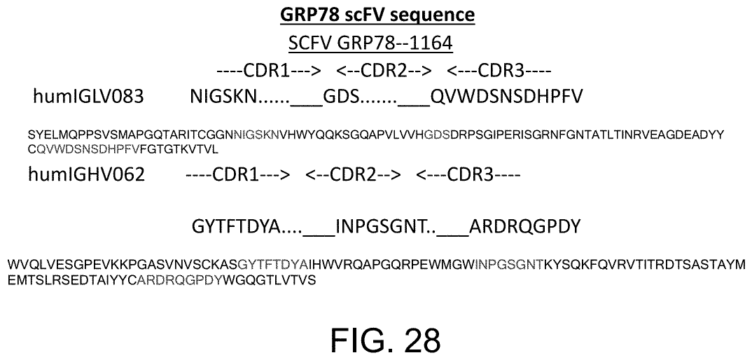

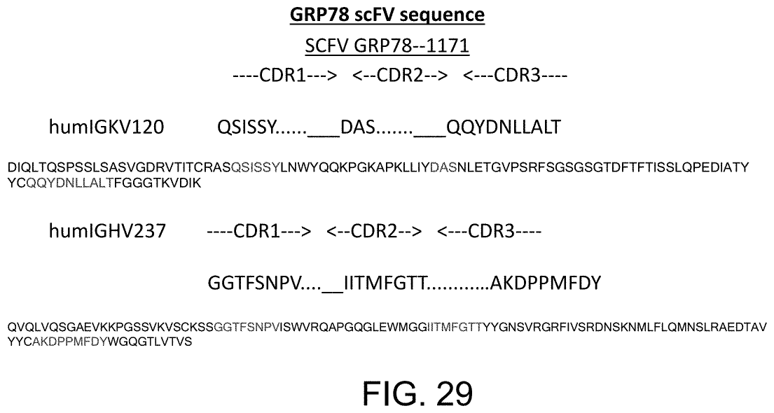

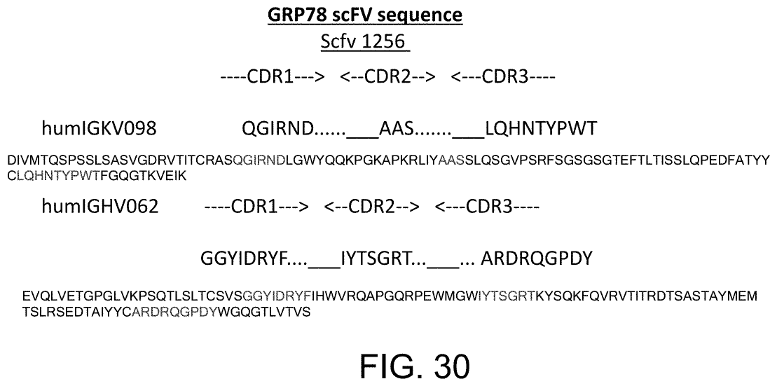

Dose-dependent effect of scFv-Fc 1171 on A549 cells using colony formation assay. Cells were seeded and irradiated with 2Gy the following day. scFv-Fc 1171 or the Fc control was added at 2 different concentrations. Another dose of 2Gy was given the next day. Colonies were counted and surviving fraction plotted. Two-way ANOVA was used for statistical analysis. *p<0.05, **p<0.01 (FIG. 23). Effect of scFv-Fc 1171 on H460 cells using colony formation assay. Cells were seeded and irradiated with 2Gy the following day. scFv-Fc 1171 or the Fc control was added at 2 different concentrations. Another dose of 2Gy was given the next day. Colonies were counted and surviving fraction plotted. A decreasing trend in surviving fraction is observed with scFvFc1171 in combination with radiation (FIG. 24).

What is claimed is:

1. An isolated antibody that specifically binds to 78-kDa glucose-regulated protein (GRP78), wherein the antibody comprises a heavy chain variable domain comprising a CDR1, CDR2, and a CDR3, wherein the heavy chain variable domain CDR1 comprises SEQ ID NO: 72, the heavy chain variable region domain CDR2 comprises SEQ ID NO:73, and the heavy chain variable region domain CDR3 selected from the group consisting of SEQ ID NO:3, 9, 23, 31, 39, and 47; and a light chain variable domain comprising a CDR1, CDR2, and CDR3, wherein the light chain variable domain CDR1 comprises SEQ ID NO:74, the light chain variable region domain CDR2 comprises SEQ ID NO:75, and the light chain variable region domain CDR3 comprises SEQ ID NO:76.

2. An isolated antibody that specifically binds to 78-kDa glucose-regulated protein (GRP78), wherein the antibody comprises a heavy chain variable domain comprising a CDR1, CDR2, and a CDR3, wherein the heavy chain variable domain CDR1 comprises SEQ ID NO: 1, the heavy chain variable region domain CDR2 comprises SEQ ID NO:2, and the heavy chain variable region domain CDR3 comprises SEQ ID NO:3; and a light chain variable domain comprising a CDR1, CDR2, and CDR3, wherein the light chain variable domain CDR1 comprises SEQ ID NO:4, the light chain variable region domain CDR2 comprises SEQ ID NO:5, and the light chain variable region domain CDR3 comprises SEQ ID NO:6.

3. An isolated antibody that specifically binds to 78-kDa glucose-regulated protein (GRP78), wherein the antibody comprises a heavy chain variable domain comprising a CDR1, CDR2, and a CDR3, wherein the heavy chain variable domain CDR1 comprises SEQ ID NO: 7, the heavy chain variable region domain CDR2 comprises SEQ ID NO:8, and the heavy chain variable region domain CDR3 comprises SEQ ID NO:9; and a light chain variable domain comprising a CDR1, CDR2, and CDR3, wherein the light chain variable domain CDR1 comprises SEQ ID NO:10, the light chain variable region domain CDR2 comprises SEQ ID NO:11, and the light chain variable region domain CDR3 comprises SEQ ID NO:12.

4. An isolated antibody that specifically binds to 78-kDa glucose-regulated protein (GRP78), wherein the antibody comprises a heavy chain variable domain comprising a CDR1, CDR2, and a CDR3, wherein the heavy chain variable domain CDR1 comprises SEQ ID NO: 21, the heavy chain variable region domain CDR2 comprises SEQ ID NO:22, and the heavy chain variable region domain CDR3 comprises SEQ ID NO:23; and a light chain variable domain comprising a CDR1, CDR2, and CDR3, wherein the light chain variable domain CDR1 comprises SEQ ID NO:24, the light chain variable region domain CDR2 comprises SEQ ID NO:25, and the light chain variable region domain CDR3 comprises SEQ ID NO:26.

5. An isolated antibody that specifically binds to 78-kDa glucose-regulated protein (GRP78), wherein the antibody comprises a heavy chain variable domain comprising a CDR1, CDR2, and a CDR3, wherein the heavy chain variable domain CDR1 comprises SEQ ID NO:29, the heavy chain variable region domain CDR2 comprises SEQ ID NO:30, and the heavy chain variable region domain CDR3 comprises SEQ ID NO:31; and a light chain variable domain comprising a CDR1, CDR2, and CDR3, wherein the light chain variable domain CDR1 comprises SEQ ID NO:32, the light chain variable region domain CDR2 comprises SEQ ID NO:33, and the light chain variable region domain CDR3 comprises SEQ ID NO:34.

6. An isolated antibody that specifically binds to 78-kDa glucose-regulated protein (GRP78), wherein the antibody comprises a heavy chain variable domain comprising a CDR1, CDR2, and a CDR3, wherein the heavy chain variable domain CDR1 comprises SEQ ID NO:37, the heavy chain variable region domain CDR2 comprises SEQ ID NO:38, and the heavy chain variable region domain CDR3 comprises SEQ ID NO:39; and a light chain variable domain comprising a CDR1, CDR2, and CDR3, wherein the light chain variable domain CDR1 comprises SEQ ID NO:40, the light chain variable region domain CDR2 comprises SEQ ID NO:41, and the light chain variable region domain CDR3 comprises SEQ ID NO:42.

7. An isolated antibody that specifically binds to 78-kDa glucose-regulated protein (GRP78), wherein the antibody comprises a heavy chain variable domain comprising a CDR1, CDR2, and a CDR3, wherein the heavy chain variable domain CDR1 comprises SEQ ID NO:45, the heavy chain variable region domain CDR2 comprises SEQ ID NO:46, and the heavy chain variable region domain CDR3 comprises SEQ ID NO:47; and a light chain variable domain comprising a CDR1, CDR2, and CDR3, wherein the light chain variable domain CDR1 comprises SEQ ID NO:48, the light chain variable region domain CDR2 comprises SEQ ID NO:49, and the light chain variable region domain CDR3 comprises SEQ ID NO:50.

8. The isolated antibody of claim 2, wherein the antibody comprises a heavy chain variable domain comprising SEQ ID NO: 13 and/or a light chain variable domain comprising SEQ ID NO: 14.

9. The isolated antibody of claim 3, wherein the antibody comprises a heavy chain variable domain comprising SEQ ID NO: 15 and/or a light chain variable domain comprising SEQ ID NO: 16.

10. The isolated antibody of claim 4, wherein the antibody comprises a heavy chain variable domain comprising SEQ ID NO: 27 and/or a light chain variable domain comprising SEQ ID NO: 28.

11. The isolated antibody of claim 5, wherein the antibody comprises a heavy chain variable domain comprising SEQ ID NO: 35 and/or a light chain variable domain comprising SEQ ID NO: 36.

12. The isolated antibody of claim 6, wherein the antibody comprises a heavy chain variable domain comprising SEQ ID NO: 43 and/or a light chain variable domain comprising SEQ ID NO: 44.

13. The isolated antibody of claim 7, wherein the antibody comprises a heavy chain variable domain comprising SEQ ID NO: 51 and/or a light chain variable domain comprising SEQ ID NO: 52.

14. The antibody of any one of claims 1-13, wherein the antibody is selected from the group consisting of a humanized antibody, a single chain variable fragment (scFv) antibody, an antibody fragment, or a chimeric antibody.

15. The antibody of any one of claims 1-14, wherein the antibody is conjugated directly or indirectly to a payload selected from the group consisting of a therapeutic agent, an imaging agent, or a combination thereof.

16. A method of enhancing radiotherapy in a subject in need thereof comprising administering an effective amount of a composition comprising an antibody that specifically binds to 78-kDa glucose-regulated protein (GRP78) such that radiotherapy is enhanced, wherein the antibody is conjugated directly or indirectly to a payload selected from the group consisting of a therapeutic agent, an imaging agent, or a combination thereof and wherein the antibody is an isolated antibody of claims 1-13.

17. The method of claim 9, further comprising administering ionizing radiation to the subject.

18. The method of claim 9, further comprising imaging the subject.

19. The method of claim 9, wherein, the therapeutic agent is one or more of a CAR-bearing immune effector cell, a BITE, or a DART.

20. A method of imaging a tumor or a cancer in a subject in need thereof, the method comprising administering a composition comprising the conjugated GRP78 to the subject, and imaging cancer in a subject, using an antibody of claims 1-13, wherein the antibody is conjugated to an imaging agent.

21. A method of treating a tumor or a cancer in a subject in need thereof comprising administering an effective amount of a composition comprising an antibody of anyone of claims 1-13, wherein the antibody is conjugated directly or indirectly to a therapeutic agent.
